(19) United States
(12) Patent Application Publication
(10) Pub. No.: US 2025/0129083 A1
(43) Pub. Date: Apr. 24, 2025

(54) IMIDAZOTETRAZINE COMPOUNDS AND TREATMENT OF TMZ-RESISTANT CANCERS

(71) Applicant: THE BOARD OF TRUSTEES OF THE UNIVERSITY OF ILLINOIS, Urbana, IL (US)

(72) Inventors: Paul J. HERGENROTHER, Champaign, IL (US); Timothy M. FAN, Mahomet, IL (US); Riley L. SVEC, Urbana, IL (US); Sydney MCKEE, Urbana, IL (US)

(73) Assignee: THE BOARD OF TRUSTEES OF THE UNIVERSITY OF ILLINOIS, Urbana, IL (US)

(21) Appl. No.: 18/834,350

(22) PCT Filed: Feb. 1, 2023
(86) PCT No.: PCT/US2023/061774

§ 371 Date: Jul. 30, 2024

Related U.S. Application Data
(60) Provisional application No. 63/305,526, filed on Feb. 1, 2022.

(51) Int. Cl. C07D 487/04; A61K 31/4188

(52) U.S. Cl. CPC C07D 487/04; A61K 31/4188

(57) ABSTRACT

Imidazotetrazines were designed to evade resistance by O⁶-methylguanine DNA methyltransferase (MGMT) while retaining suitable hydrolytic stability, allowing for effective prodrug activation and biodistribution. One compound, called TABZ, exhibits activity against cancer cells irrespective of MGMT expression and MMR status. TABZ has comparable blood-brain barrier penetrance and comparable hematological toxicity relative to TMZ, while also matching its maximum tolerated dose (MTD) in mice when dosed once-per-day over five days. The activity of TABZ is independent of the two principal mechanisms suppressing the effectiveness of TMZ, making it a promising new candidate for the treatment of GBM, especially those that are TMZ resistant.

### RELATED APPLICATIONS

This application claims priority under 35 U.S.C. § 119(e) to U.S. Provisional Patent Application No. 63/305,526, filed Feb. 1, 2022, which is incorporated herein by reference.

### GOVERNMENT SUPPORT

This invention was made with government support under Grant No. R01-CA256481 awarded by the National Institutes of Health. The government has certain rights in the invention.

### BACKGROUND OF THE INVENTION

The prognosis of patients with glioblastoma (GBM), the most prevalent and malignant type of primary brain tumor, is extremely poor. GBM is incurable and without treatment median survival of these patients is less than three months. The standard treatment regimen for GBM patients consists of bulk surgical resection and radiotherapy (RT) with concomitant and adjuvant temozolomide (TMZ), a small molecule DNA alkylating agent. TMZ is an imidazotetrazine prodrug that undergoes hydrolytic ring opening under physiological conditions (t_{1/2}˜₂-1.5-2 hours in human patients), releasing methyl diazonium ion, the active DNA alkylating species in addition to 5-aminoimidazole-4-carboxamide (AIC). The cytotoxicity of the methyl diazonium ion is derived from methylation of the O⁶ position of guanine residues, which triggers futile cycles of mismatch repair (MMR)-mediated DNA repair and subsequent DNA double strand breaks. The addition of TMZ to RT improves overall patient survival to 15 months (up to an 8 month survival benefit vs RT alone) which has cemented its use in the clinic since it gained frontline approval in 2005 for GBM patients.

It is well-known that certain GBM patient populations do not respond to TMZ therapy. Expression of O⁶-methylguanine DNA methyltransferase (MGMT), an alkyltransferase responsible for the direct repair of O⁶-methylguanine DNA adducts, serves as the primary source of intrinsic resistance to TMZ. Silencing of the MGMT gene through promoter methylation, a common epigenetic phenomenon in gliomas, is strongly correlated with a therapeutic response to TMZ and an increased survival rate, but 55-60% of GBMs have MGMT unmethylated promoters. Patients bearing these MGMT-positive tumors have median survival times 11 months less than those with MGMT-negative tumors when treated with TMZ. MGMT promoter methylation status is routinely used as a biomarker for GBM patients but is not implemented to stratify therapeutic decision-making; patients with both MGMT methylated and unmethylated promoters are treated with TMZ, even though there is virtually no benefit in MGMT unmethylated patients.

The other major clinical mode of resistance to TMZ is loss of MMR function, as proper MMR is required to trigger the futile processing of O⁶-methylguanine lesions. A defective MMR system leads to tumoral tolerance of the O⁶-methylguanine adducts generated by TMZ, global G:C→A:T transition mutations, and more malignant hypermutated tumors. Approximately 25% of recurrent GBM tumors harbor inactivating mutations in the MMR pathway (namely in the MSH6, MSH2, MLH1, or PMS2 genes). Distressingly, complete inactivation of MMR is not required to bestow resistance to TMZ, and even a 15% reduction in MMR protein expression can result in insensitivity to the drug. The frequency of these two resistance mechanisms (MGMT expression and loss of MMR function) precludes a majority of GBM patients, both newly diagnosed and recurrent, from deriving any benefit from TMZ.

MGMT is a suicide enzyme that loses enzymatic activity upon methylation and is subsequently targeted for degradation by the proteasome. Therefore, several strategies to deplete tumoral MGMT and thus regain sensitivity to TMZ have been explored. One approach is to alter the dosing regimen of TMZ to achieve continuous drug exposure and maintain alkylated (inactive) MGMT to perpetuate chemotherapy sensitization. Prolonged inhibition of MGMT expression or activity has been measured in peripheral blood mononuclear cells taken from patients treated with dose-dense TMZ (e.g. 21/28 days vs. 5/28 days), but ensuing clinical trials have reported no differences in efficacy when compared to the standard 5/28 day protocol. Another approach is to co-administer TMZ together with an MGMT inhibitor. The most widely used MGMT inhibitor is pseudosubstrate O⁶-benzylguanine (O6BG, reviewed by Rabik et al.), which potently inhibits MGMT activity. Despite potentiation of the cytotoxicity of TMZ in MGMT-expressing cell lines and murine tumor models, no clinical benefit has been observed from this combination or from the combination of TMZ with other MGMT inhibitors.

Instead of attempting to deplete tumoral MGMT, a distinct strategy would be to create an imidazotetrazine analogue that delivers a moiety other than methyl, which would be irremovable by MGMT. These ‘MGMT-independent’ compounds would ideally retain the favorable properties of TMZ (aqueous prodrug activation, blood-brain barrier (BBB) permeability, etc.), and serve as a treatment option for all GBM patients irrespective of MGMT status. Previous attempts to employ this strategy using imidazotetrazines with a variety of alkyl groups at the N3 position presume transfer of the alkyl group to DNA; however, obtaining an N3-substituted analogue that retains activity in MGMT-positive cell lines and demonstrates proper hydrolytic stability has proven elusive, so this strategy remains untested in vivo.

One possible obstacle for an MGMT-independent imidazotetrazine is the potential for enhanced in vivo toxicity, as MGMT functions as a systemic protectant against the cytotoxic and mutagenic effects of O⁶-methylguanine, and therefore mitigates the toxic effects of TMZ in non-malignant tissues. As a result, when TMZ was co-administered with O6BG in the clinic, the cumulative TMZ dose had to be reduced by 50-75% due to exacerbated myelotoxicity. These dose reductions have been blamed for the disappointing clinical results of TMZ/MGMT inhibitor combinations.

Accordingly, there is a need for improved compounds that possess desirable properties of TMZ but are not cancer resistant, have better brain penetration, lower toxicity, and provide improved patient survival rates.

### SUMMARY

Herein, we report the discovery of a novel imidazotetrazine, the compound CPZ, that evades both MGMT and MMR-mediated resistance. To minimize on-target but off-tumor myelosuppressive effects, CPZ was designed to have enhanced localization to the central nervous system (CNS). As reported herein, CPZ not only outperforms TMZ in culture with cell lines expressing MGMT and lacking functional MMR, but also shows an increase in BBB penetrance and noninferior hematological toxicity relative to TMZ. CPZ represents a new therapeutic alternative to TMZ, particularly for the large GBM patient population (primary and recurrent) that possesses MGMT-positive and/or MMR-deficient tumors. The compounds detailed herein can now be used to directly test the hypothesis that imidazotetrazines delivering alternative alkyl groups can be effective against TMZ-resistant brain tumors in vivo.

Accordingly, this disclosure provides a compound of Formula I:

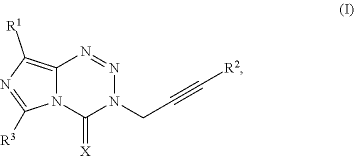

or a salt thereof,
wherein
   X is O or S;
   R¹ is —C(═O)NR^{b}R^{c}, F, Br, or I; wherein
      R^{b} is —(C₁-C₆)alkyl, —(C₃-C₆)cycloalkyl, or H;
      R^{c} is —(C₁-C₆)alkyl, —(C₃-C₆)cycloalkyl, or H;
   R² is —(C₁-C₆)alkyl, —(C₃-C₆)cycloalkyl, or H; and
   R³ is H, halo, —(C₁-C₆)alkyl, or —(C₃-C₆)cycloalkyl;
wherein each —(C₁-C₆)alkyl or —(C₃-C₆)cycloalkyl are optionally substituted with one or more substituents; and
   each —(C₁-C₆)alkyl is optionally partially or fully unsaturated, and unbranched or branched.

This disclosure also provides a method of treating a cancer comprising, administering to a subject diagnosed with cancer a therapeutically effective amount of a compound disclosed above, or a composition thereof, wherein the cancer is thereby treated.

The invention provides novel compounds of Formula I and Formula II, intermediates for the synthesis of compounds of Formula I and Formula II, as well as methods of preparing compounds of Formula I and II. The invention also provides compounds of Formula I and II that are useful as intermediates for the synthesis of other useful compounds. The invention provides for the use of compounds of Formula I and Formula II for the manufacture of medicaments useful for the treatment of bacterial infections in a mammal, such as a human.

The invention provides for the use of the compositions described herein for use in medical therapy. The medical therapy can be treating cancer, for example, brain cancer, breast cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Selection and evaluation of MGMT-independent imidazotetrazines. (A) IC₅₀ values (M, 7 days) of TMZ and compound 10 in an expanded panel of GBM cell lines. Cell viability was assessed via the Alamar Blue assay, n≥3, error is SEM. (B) Hydrolytic stability of TMZ at pH 7, 7.4, and 8. Percentage of prodrug remaining was quantified by HPLC, n≥2. (C) Hydrolytic stability of compound 10 at pH 7, 7.4, and 8. Percentage of prodrug remaining was quantified by HPLC, n≥2.

FIG. 2A-F. (A) 7 day IC₅₀ values (M) of compound 10 and CPZ in an expanded panel of GBM cell lines. Cell viability was assessed via the Alamar Blue assay, n≥3, error is SEM. Data for compound 10 is the same as shown in FIG. 1A. (B-E) 7 day dose-response curves of TMZ and CPZ in cell lines with variable MGMT and MMR status. Error is SEM, n≥3. (F) Killing kinetics of CPZ compared to TMZ in the U87 cell line. Cells were treated with compound once and viability was assessed every 24 hours via Alamar Blue assay. Error is SEM, n≥2.

FIG. 3A-E. Investigation of the mechanism of action and efficacy of CPZ in MGMT+ cell lines. (A) Detection of O⁶-methyl-2′-deoxyguanosine or O⁶-propargyl-2′-deoxyguanosine in GL261 cells after treatment with TMZ or CPZ, respectively. GL261 cells were treated with the indicated concentration of compound for 8 hours before they were harvested and genomic DNA was extracted. DNA (10 μg) was hydrolyzed and submitted to LC-MS/MS for quantitation. Both O⁶-Me-dG and O⁶-proparyl-dG were below the limit of detection in the DMSO control. Error is SEM, n≥3. Cytotoxicity of TMZ (B) or CPZ (C) in T98G cells pretreated (3 hours) with MGMT inhibitor O6BG. Error is SEM, n≥3. (D) 7 day dose-response curves of TMZ and CPZ in GL261 cells. Error is SEM, n≥3. (E) 7 day dose-response curves of TMZ and CPZ in GL261 MGMT+ cells. Error is SEM, n≥3.

FIG. 4A-F. In vivo biodistribution and hematological toxicity studies with CPZ. (A,B) Serum and brain concentrations of TMZ and CPZ 15 min after administration of 25 mg/kg compound (IV) to mice. Number of mice per cohort ≥3. (C) Brain:serum ratios using data from (A,B). (D-F) White blood cell, lymphocyte, and neutrophil counts in mice 7 days after administration of a single 125 mg/kg IV dose of TMZ and CPZ. Statistical significance was determined by using a two-sample Student's t-test (two-tailed test, assuming equal variance). *P<0.05, **P<0.01.

FIG. 5A-C. Blood-Brain Barrier (BBB) Penetration Comparison of TABZ to TMZ (A-C). TMZ and TABZ were formulated in 20% DMSO in PBS at 5 mg/ml, 100 uL injection. Female CD-1 mice were treated with 25 mg/kg of compounds via lateral tail iv injection. At each time point, the mice were humanely sacrificed and blood was collected, and the brain was perfused with saline before it was harvested. Upon collection brain and serum samples were acidified. Brain was homogenized with tissue homogenizer in H₃PO₄ solution. Students' T-test: * p<0.05 ** p<0.01. *** p<0.001, **** p<0.0001, n.s.: not significant. TABZ is tolerated at 66 mg/kg×2/day over 5 days via IP injection and 50 mg/kg×1/day via IV injection (comparable to TMZ).

FIG. 6A-C. Comparison of formulations for TABZ BBB penetrance (A-C). Systems Oncology (SO) formulation: 7/10/20/63 EtOH/PG/PEG400/Kolliphor EL (10% in water); Original Formulation (OF): 15/15/70 DMSO/PG/PBS. Compounds were formulated at 2.5 mg/mL and delivered in a 200 uL injection, 25 mg/kg iv for TABZ.

FIG. 7A-D. TABZ versus TMZ activity in MGMT Proficient/MMR Deficient Cells (A-D).

FIG. 8A-C. Rapid Cancer Cell Death Phenotype. TABZ and CPZ induce cell death quickly relative to TMZ and suggests MMR independency (A-C).

FIG. 9A-D. Comparing the Hematological Toxicity of TABZ and TMZ (A-D). Mice were treated with either vehicle (15/15/70 DMSO/PG/PBS), TMZ (50 mg/kg), or TABZ (50 mg/kg)1×/day over 5 days intravenously (200 uL) via lateral tail vein injection. Mice were sacrificed on day 8 for blood and tissue analysis.

FIG. 10A-B. Syngeneic Subcutaneous Model (A, B). TABZ and TMZ in vivo: C57BL/6 mice were inoculated with 1×10⁶ cells and treated xl/day over 5 days with either vehicle, TMZ, or TABZ (via IV, 50 mg/kg, starting on day 5). TMZ mice showed an exacerbation of tumor growth, likely as a result of its hematological toxicity and immune suppression, whereas TABZ showed no exacerbation.

### DETAILED DESCRIPTION

Glioblastoma (GBM) is the most lethal primary brain tumor. Currently, frontline treatment for primary GBM includes the DNA-methylating drug temozolomide (TMZ, of the imidazotetrazine class), while the optimal treatment for recurrent GBM remains under investigation. Despite its widespread use, a majority of GBM patients do not respond to TMZ therapy; expression of the O⁶-methylguanine DNA methyltransferase (MGMT) enzyme and loss of mismatch repair (MMR) function as the principal clinical modes of resistance to TMZ. Here we describe a novel imidazotetrazine designed to evade resistance by MGMT while retaining suitable hydrolytic stability, allowing for effective prodrug activation and biodistribution. This dual-substituted compound, called CPZ, exhibits activity against cancer cells irrespective of MGMT expression and MMR status. CPZ has greater blood-brain barrier penetrance and comparable hematological toxicity relative to TMZ, while also matching its maximum tolerated dose (MTD) in mice when dosed once-per-day over five days. The activity of CPZ is independent of the two principal mechanisms suppressing the effectiveness of TMZ, making it a promising new candidate for the treatment of GBM, especially those that are TMZ resistant.

Additional information and data supporting the invention can be found in the following publication: WO 2020/033880, published Feb. 13, 2020, and ACS Chem. Biol. 2022, 17(2), 299-313, which publications are incorporated herein by reference.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R.J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated.

Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modem Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl(iso-propyl), 1-butyl, 2-methyl-1-propyl(isobutyl), 2-butyl(sec-butyl), 2-methyl-2-propyl(t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences on one carbon atom or one on each of two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10-membered, more preferably 4 to 7-membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aromatic” refers to either an aryl or heteroaryl group or substituent described herein. Additionally, an aromatic moiety may be a bisaromatic moiety, a trisaromatic moiety, and so on. A bisaromatic moiety has a single bond between two aromatic moieties such as, but not limited to, biphenyl, or bipyridine. Similarly, a trisaromatic moiety has a single bond between each aromatic moiety.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R‘ wherein R’ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as 0, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “IC₅₀” is generally defined as the concentration required to inhibit a specific biological or biochemical function by half, or to kill 50% of a sample of cells, in a designated time period, typically 24 hours.

### Embodiments of the Technology

This disclosure provides a compound of Formula I:

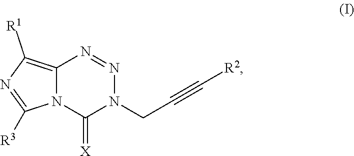

or a salt thereof,
wherein
   X is O or S;
   R¹ is —C(═O)NR^{b}R^{c}, F, Br, or I; wherein
      R^{b} is —(C₁-C₆)alkyl, —(C₃-C₆)cycloalkyl, or H;
      R^{c} is —(C₁-C₆)alkyl, —(C₃-C₆)cycloalkyl, or H;
   R² is —(C₁-C₆)alkyl, —(C₃-C₆)cycloalkyl, or H; and
   R³ is H, halo, —(C₁-C₆)alkyl, or —(C₃-C₆)cycloalkyl;
wherein each —(C₁-C₆)alkyl or —(C₃-C₆)cycloalkyl are optionally substituted with one or more substituents; and
   each —(C₁-C₆)alkyl is optionally partially or fully unsaturated, and unbranched or branched.

In various embodiments, X is O. In various embodiments, R¹ is —C(═O)—N[(C₁-C₆)alkyl]₂. In various embodiments, R¹ is —C(═O)—NH(C₁-C₆)alkyl. In various embodiments, R² is CH₃. In various embodiments, R² is H. In various embodiments, R3 is H.

In some embodiments, the compound of Formula I is a compound of Formula II:

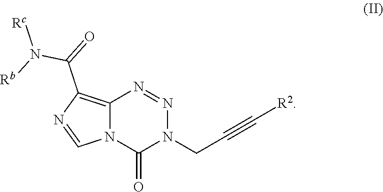

In various embodiments, R^{b} is —(C₁-C₆)alkyl, R is —(C₁-C₆)alkyl, and R² is —(C₁-C₆)alkyl. In various embodiments, R^{b}, R^{c} and R³ are CH₃.

In some embodiments the compound is:

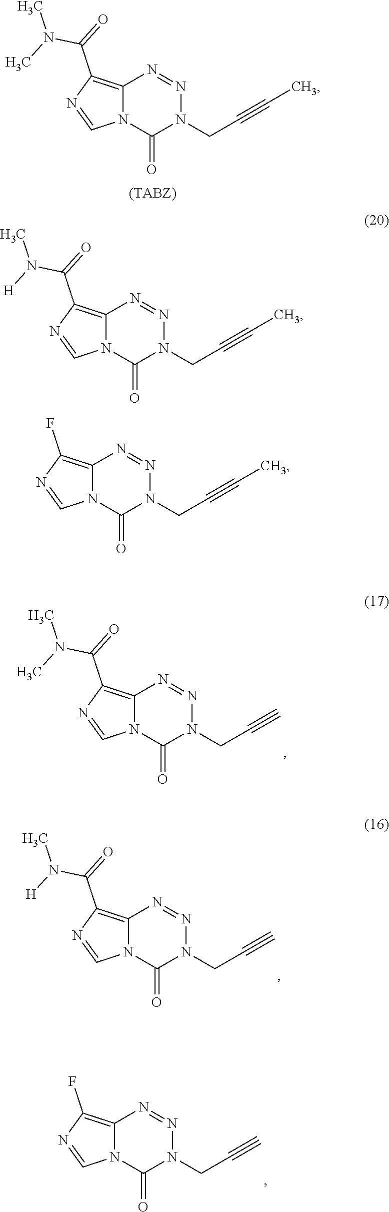 [SMILES: CC#CCn1nnc2c(C(=O)N(C)C)ncn2c1=O ; CC#CCn1nnc2c(C(=O)NC)ncn2c1=O ; CC#CCn1nnc2c(F)ncn2c1=O ; C#CCn1nnc2c(C(=O)N(C)C)ncn2c1=O ; C#CCn1nnc2c(C(=O)NC)ncn2c1=O ; C#CCn1nnc2c(F)ncn2c1=O]

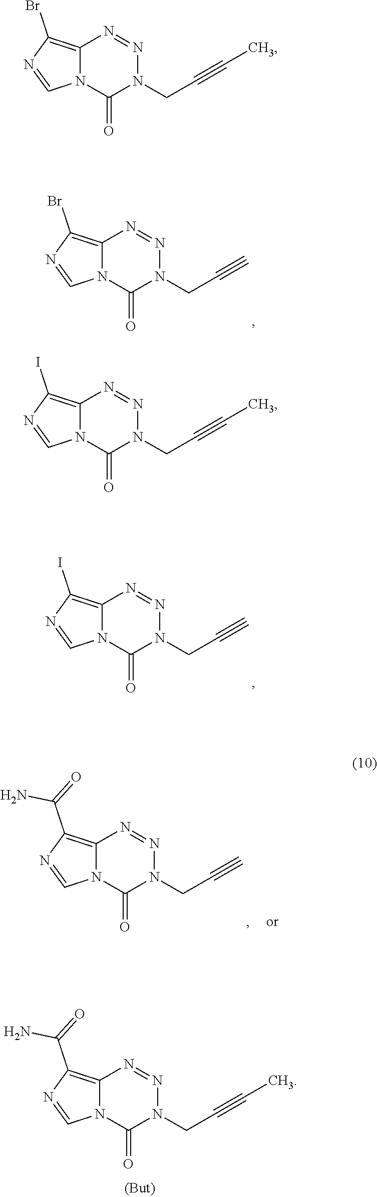 [SMILES: CC#CCn1nnc2c(Br)ncn2c1=O ; C#CCn1nnc2c(Br)ncn2c1=O ; CC#CCn1nnc2c(I)ncn2c1=O ; C#CCn1nnc2c(I)ncn2c1=O ; C#CCn1nnc2c(C(N)=O)ncn2c1=O ; CC#CCn1nnc2c(C(N)=O)ncn2c1=O]

In some embodiments the compound is:

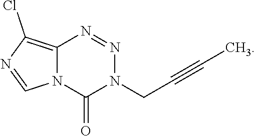 [SMILES: CC#CCn1nnc2c(Cl)ncn2c1=O]

Also, this disclosure provides a composition comprising a compound disclosed herein and a pharmaceutically acceptable excipient.

Additionally, this disclosure provides a method of treating a cancer, or a compound disclosed herein for use in a method of treating cancer, comprising administering to a cancer subject in need of cancer therapy a therapeutically effective amount of a compound disclosed herein, or a therapeutically effective amount of a composition of a compound disclosed herein, wherein the cancer is thereby treated.

In various embodiments, the dosing is once per day, twice per day, three times per day, four times per day, or more than four times per day. In some embodiments, a compound disclosed herein and a second therapeutic agent is administered to the subject sequentially or simultaneously.

In some embodiments, the second agent is atezolizumab, bevacizumab, bortezomib, carboplatin, carmustine, chlorambucil, cisplatin, cyclophosphamide, dabrafenib, dactinomycin, daratumumab, doxorubicin, enzalutamide, encorafenib, entrectinib, etoposide, fludarabine, fluorouracil, fulvestrant, gefitinib, ibrutinib, ifosfamide, imatinib, lenalidomide, nivolumab, obinutuzumab, oxaliplatin, paclitaxel, palbociclib, pembrolizumab, pertuzumab, rituximab, ruxolitinib, sorafenib, tamoxifen, temozolomide, thiotepa, topotecan, trametinib, trastuzumab, vemurafenib, venetoclax, vinblastine, or vincristine.

In some embodiments, the second agent is for the treatment of a brain cancer or tumor, wherein the second agent is belzutifan, bevacizumab, carmustine, everolimus, lomustine, naxitamab, or temozolomide.

In some embodiments, the cancer is melanoma, leukemia, breast cancer, lung cancer, pancreatic cancer, prostate cancer, colon cancer or brain cancer. In various embodiments, the cancer is brain cancer. In various embodiments, the cancer is glioblastoma (GBM). In other embodiments, the cancer is a glioma, meningioma, pituitary adenoma, or a nerve sheath tumor.

In some embodiments the brain cancer or brain tumor is anaplastic astrocytoma, anaplastic oligodendroglioma, astrocytoma, central neurocytoma, choroid plexus carcinoma, choroid plexus papilloma, choroid plexus tumor, colloid cyst, dysembryoplastic neuroepithelial tumor, ependymal tumor, fibrillary astrocytoma, giant-cell glioblastoma, glioblastoma multiforme, gliomatosis cerebri, gliosarcoma, hemangiopericytoma, medulloblastoma, medulloepithelioma, meningeal carcinomatosis, neuroblastoma, neurocytoma, oligoastrocytoma, oligodendroglioma, optic nerve sheath meningioma, pediatric ependymoma, pilocytic astrocytoma, pinealoblastoma, pineocytoma, pleomorphic anaplastic neuroblastoma, pleomorphic xanthoastrocytoma, primary central nervous system lymphoma, sphenoid wing meningioma, subependymal giant cell astrocytoma, subependymoma, trilateral retinoblastoma.

In various embodiments, a dose of a compound or composition disclosed herein is administered is about 0.5 mg/kg to about 100 mg/kg of body weight per day. In other embodiments, the number of milligrams of the compound or composition per kilogram of body weight in a dose is about: 1, 5, 10, 15, 20, 25, 30, 35, 40, 45, 50, 55, 60, 65, 70, 75, 80. 85, 90, 95, 100, 110, 120, 130, 140, 150, 160, 170, 180, 190, 200, 225, 250, 275, 300, 350, 400, 500, or 1000.

In various embodiments, a dose of a compound or composition disclosed herein is administered 5 mg/m² to 1000 mg/m² of body surface area. In other embodiments, the number of milligrams of the compound or composition per m² of body surface area in a dose is about: 1, 5, 10, 15, 20, 25, 30, 35, 40, 45, 50, 55, 60, 65, 70, 75, 80. 85, 90, 95, 100, 110, 120, 130, 140, 150, 160, 170, 180, 190, 200, 225, 250, 275, 300, 350, 400, 500, or 1000.

In various embodiments, a unit dose of a compound or composition disclosed herein is administered is 5 mg to 1000 mg. In other embodiments, the number of milligrams of the compound or composition in a unit dose is about: 1, 5, 10, 15, 20, 25, 30, 35, 40, 45, 50, 55, 60, 65, 70, 75, 80. 85, 90, 95, 100, 110, 120, 130, 140, 150, 160, 170, 180, 190, 200, 225, 250, 275, 300, 350, 400, 500, or 1000.

In various embodiments, a therapeutically effective concentration of a compound or composition disclosed herein that is contacting the cancer is about 1 nM to about 10 μM. In other embodiments, the therapeutically effective concentration (in nM or μM) of the compound or composition that is contacting the cancer is about: 1, 5, 10, 15, 20, 25, 30, 35, 40, 45, 50, 55, 60, 65, 70, 75, 80. 85, 90, 95, 100, 110, 120, 130, 140, 150, 160, 170, 180, 190, 200, 225, 250, 275, 300, 350, 400, 500, or 1000.

In various embodiments, a dose of a compound or composition disclosed herein is administered once per day, twice per day, or thrice per day. In various embodiments, a compound or composition disclosed herein is administered orally, intravenously, or intracranially.

In various embodiments, the compound is 3-(but-2-yn-1-yl)-N,N-dimethyl-4-oxo-3,4-dihydroimidazo[5,1-d][1,2,3,5]tetrazine-8-carboxamide (“TABZ”).

In various embodiments, a compound disclosed herein is used in therapy, or used as a medicament. In various embodiments, a compound disclosed herein is used in the treatment of a cancer. Also, this disclosure provides use of a compound disclosed herein in the manufacture of a medicament for treating a cancer.

### Results.

Design and Evaluation of N3-substituted Imidazotetrazines. The substituents known to be removable from the O⁶ position of guanine by MGMT extend beyond methyl and include ethyl, chloroethyl, and benzyl. As such, a panel of imidazotetrazines was synthesized containing diverse functionality at the N3 position beyond simple alkyl groups (Table 1). To identify candidate compounds exhibiting anticancer activity independent of MGMT expression, the U87 (no MGMT expression) and T98G (high MGMT expression) GBM cell lines were chosen for an initial screen. TMZ has an IC₅₀ value of 50 μM in U87 cells, but exhibits >10-fold loss of activity in the T98G cell line (IC₅₀>550 μM) (Table 1), attributable to MGMT-mediated resistance.

As a control, imidazotetrazine 1, possessing no alkylating moiety at N3, was constructed and was unable to induce cell death (Table 1). Consistent with the literature (J Med Chem 1995, 38, 1493), ethyl variant 2 demonstrated differential activity in the presence and absence of MGMT but was much less active than TMZ, presumably due to the competing P-elimination pathway possible for the ethyl but not methyl diazonium ion; strong evidence for this hypothesis comes from NMR studies that suggest the formation of ethene from 2 in aqueous media. Reduced activity was also observed for higher alkyl analogue 3. First-generation imidazotetrazine mitozolomide (MTZ) possessed robust cytotoxicity against U87 and T98G cells but is a known DNA cross-linker. Sterically-demanding isopropyl (4), tert-butyl (5), and neopentyl (6) groups were incorporated to potentially disfavor the S_{N}2-mediated dealkylation pathway utilized by MGMT. Each of these compounds, however, were found to be inactive in cells (Table 1), perhaps due to a challenging DNA alkylation event from bulky diazonium ions.

TABLE 1
Seven-day IC₅₀ values (μM) of N3-substituted imidazotetrazines in the U87 and T98G GBM
cell lines. Error is standard error of the mean (SEM), n ≥ 3.

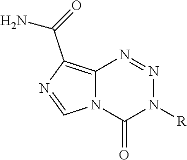

| | | MGMT (−) | MGMT (+) |
|---|---|---|---|
| Compound | R | U87 | T98G |
| TMZ | Me | 50 ± 20 | 570 ± 20 |
| 1 | H | >1000 | >1000 |
| 2 | Et | 470 ± 100 | >1000 |
| 3 | n-Pr | >1000 | >1000 |
| MTZ | —CH₂CH₂Cl | 30 ± 6 | 85 ± 9 |
| 4 | i-Pr | >1000 | >1000 |
| 5 | t-Bu | 570 ± 30 | >1000 |
| 6 | —CH₂C(CH₃)₃ | >1000 | >1000 |
| 7 | 4-OMe—Ph | 45 ± 10 | 60 ± 10 |
| 8 | 4-NMe₂-—Ph | 24 ± 2 | 44 ± 6 |
| 9 | —CH₂CH═CH₂ | 220 ± 60 | 320 ± 10 |
| 10 | —CH₂C≡CH | 29 ± 6 | 77 ± 7 |
| 11 | —CH₂OPh | 21 ± 3 | 35 ± 1 |
| 12 | —CH₂NHCOPh | 240 ± 20 | 580 ± 100 |

Imidazotetrazines 7 and 8 were designed with electron-rich arenes at the N3 position. Presumably, these compounds are precursors to arene diazonium ions, which could generate DNA species for which S_{N}2 chemistry is not feasible. Interestingly, both 7 and 8 exhibit a desirable cytotoxicity profile as each inhibits GBM cell growth with IC₅₀ values <100 μM in both cell lines (Table 1), suggestive of MGMT-independent activity. Notably, arene diazoniums exhibit a wide array of aryl transfer activity in biological systems including direct protein labeling of electron-rich side chains, particularly tyrosine and cysteine; this phenomenon, coupled with their significantly higher cLogP compared to TMZ (Table 2), prompted us to eliminate these derivatives from further advancement. Allyl derivative 9 is not exceptionally active, but the installation of a propargyl group, employed frequently for labeling studies in biological systems due to its biorthogonality, imbued 10 with a promising profile (Table 1); activity in the presence of MGMT has been previously observed for compound 10. Embedded ether-containing imidazotetrazine 11 also exhibits MGMT-independent phenotype with potent IC₅₀ values <40 μM in each cell line, but a similar compound with an embedded amide (12) does not provide the same degree of activity (Table 1).

TABLE 2
Examples of N3-substituted imidazotetrazines that demonstrate
MGMT-independent anticancer activity and their relative
BBB penetrance predicted using cLogBB values and CNS
MPO scores. Molecular properties were calculated using
the Chemicalize platform (ChemAxon).

| Compound | TMZ | 7 | 10 | 11 |
|---|---|---|---|---|
| MW | 194.15 | 286.25 | 218.18 | 286.25 |
| cLogP | −0.811 | 1.327 | −0.711 | 0.627 |
| cLogBB | −1.47 | −1.38 | −1.44 | −1.34 |
| MPO | 5.0 | 4.7 | 5.0 | 4.7 |

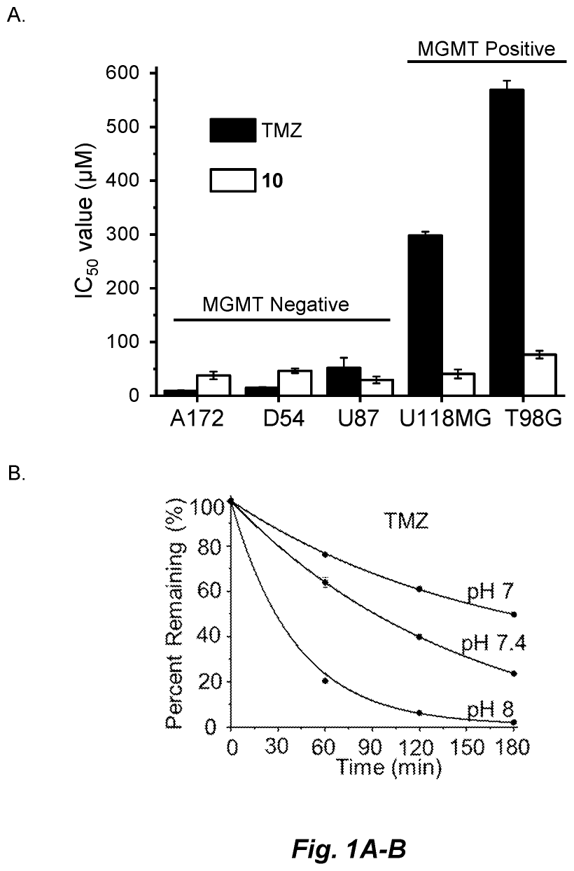

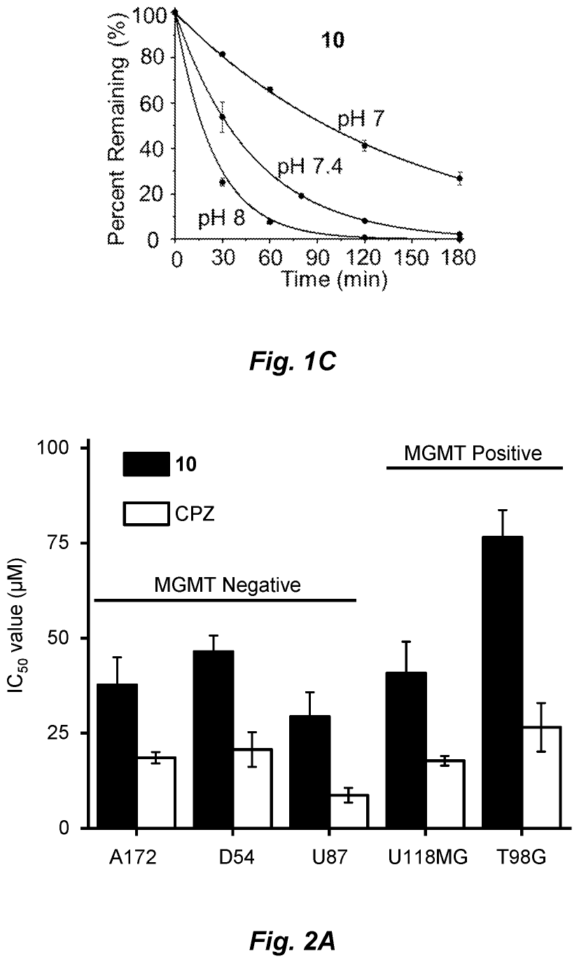

N3-propargyl compound 10 was selected for further development because of its small size and comparable cLogP to TMZ (Table 2). Compound 10 also possesses equivalent cLogBB and CNS MPO scores to TMZ, suggestive of analogous BBB permeability (Table 2). The anticancer activity of 10 was assessed against an expanded panel of GBM cell lines with or without MGMT expression (FIG. 1A). Cellular resistance conferred by MGMT is apparent for TMZ as there is >6-fold difference in IC₅₀ values between MGMT-negative and positive cell lines. Conversely, no such differential profile is observed for compound 10 as all IC₅₀ values are <100 μM, suggesting that compound cytotoxicity is not affected by MGMT expression. Despite promising activity against GBM cells in culture, 10 is known to have limited efficacy in an intracranial efficacy model. This discrepancy prompted a closer examination of the most critical feature of the imidazotetrazine class of molecules, hydrolytic stability. TMZ spontaneously hydrolyzes in aqueous solution with a half-life of ˜1.5-2 hours in vitro (PBS, pH 7.4, 37° C.) and in vivo (human pharmacokinetics).

An assay was recently reported that permits the comparison of C8-substituted imidazotetrazines by quantifying the percentage of prodrug remaining after a 2 hour timepoint. As expected, the hydrolytic stability of TMZ was strongly dependent on pH, and TMZ has a stability of 40% remaining after 2 hours at pH 7.4 (FIG. 1B). Compared to TMZ, compound 10 exhibits a much faster (but still pH-dependent) rate of hydrolysis with only 8% remaining after 2 hours at pH 7.4 (FIG. 1C). This finding was reinforced when the half-life of 10 measured in PBS (pH 7.4, 37° C.) was found to be ˜30 min compared to ˜90 min for TMZ (Table 3). Thus, the observed inability of 10 to elicit an anticancer effect and prolong survival in an intracranial murine model of GBM may be attributed to rapid compound hydrolysis prior to reaching the brain and alkylating tumoral DNA.

TABLE 3
Half-lives of TMZ and 10 calculated from the hydrolytic stability data.

| | Half-life (min) | | |
|---|---|---|---|
| Compound | pH 7 | pH 7.4 | pH 8 |
| TMZ | 178 ± 5 | 92 ± 4 | 29 ± 1 |
| 10 | 97 ± 5 | 34 ± 5 | 15 ± 1 |

Tuning Hydrolytic Stability and the Identification of CPZ. Fortunately, a recent report revealed that the hydrolytic stability of imidazotetrazines can be tuned by modifying the substituent at the C8 position, and these modifications do not disturb the alkylating capacity of the resultant prodrugs but may enhance drug pharmacokinetics and/or BBB penetrance. According to the trend derived between the electronics of the C8 substituent (measured by the σₚ value) and prodrug stability, replacing the primary amide at the C8 position of 10 with a less electron-withdrawing substituent than the primary amide (σₚ=0.36), should confer greater aqueous stability; indeed, replacement of the amide of 10 with a variety of functional groups imparted greater hydrolytic stability due to this electronic effect, as seen with the secondary amide (13, σₚ=0.36), tertiary amide (14, σₚ=0.28), and chloro(15, σₚ=0.23) derivatives (Scheme 1).

Scheme 1. Design and evaluation of the dual-substituted imidazotetrazine CPZ. Strategy used to generate a hydrolytically-stable, MGMT-independent imidazotetrazine. Tuning the stability of compound 10 via the C8 position led to novel dual-substituted compounds, including CPZ. Stability values represent the percentage of parent prodrug remaining after 2 hour incubation in PBS (pH 7.4, 37° C.). The hydrolytic stability for TABZ is 31±1% after 2 h, and for compound 20 (e.g., N-Me derivative) the hydrolytic stability is 11±1% after 2 h.

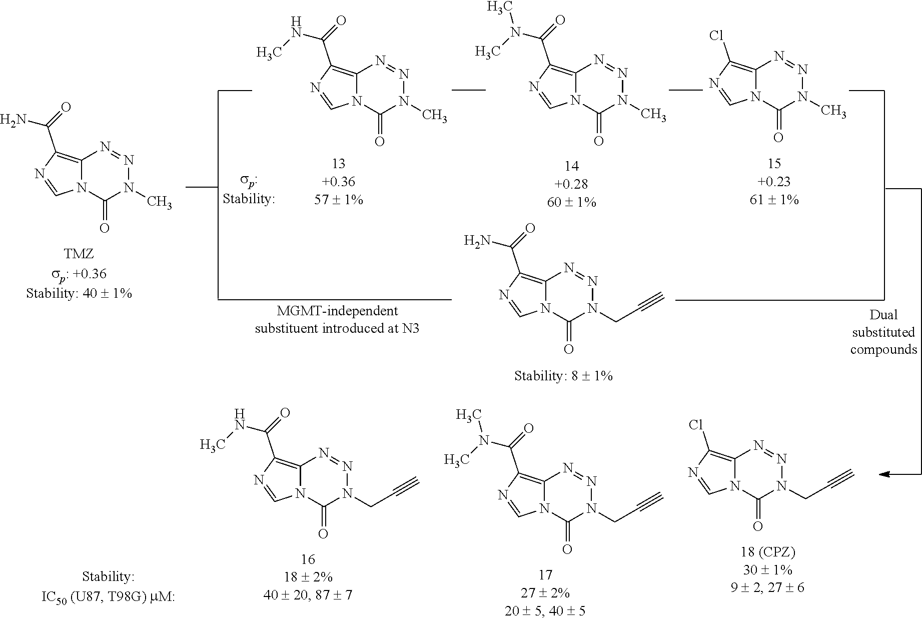

As shown in Scheme 1, the stabilizing substitutions at C8 were combined with the MGMT-evading substituent at N3 to give novel dual-substituted imidazotetrazines. All three compounds (16, 17, 18, Scheme 1) appear very promising; chloro-substituted compound 18 (dubbed CPZ) was prioritized for further assessment due to its low molecular weight and the stability of the C8-chloro to oxidative metabolism. Tuning the hydrolytic stability with a C8-chloro modification significantly improves the 2-hour stability from 8% (for 10) to 30% (for CPZ) at pH 7.4, 37° C. (Scheme 1). The hydrolytic stability remained pH-dependent and CPZ demonstrated a half-life of 72 min at pH 7.4. The other two dual-substituted compounds, 16 and 17, also demonstrated an improved two-hour stability (18% and 27%, respectively, at pH 7.4, 37° C.). All three derivatives demonstrated anticancer activity in both MGMT- and MGMT+ cell lines, with CPZ being the most potent (Scheme 1).

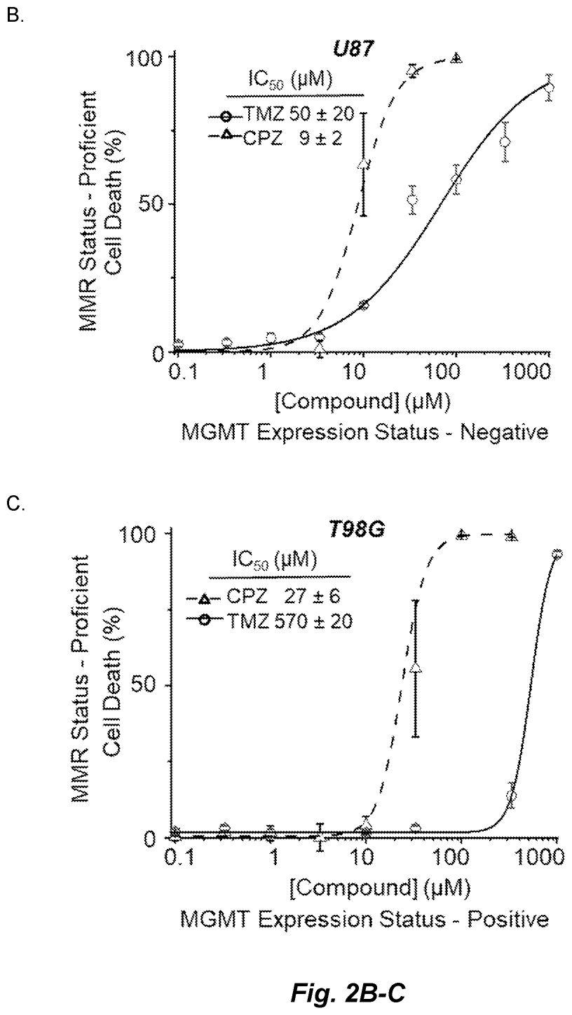

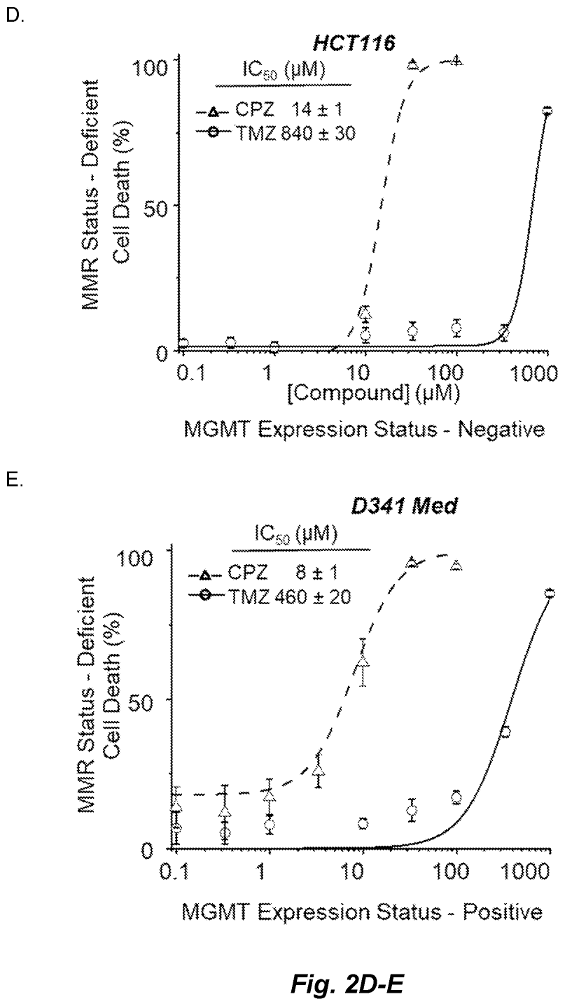

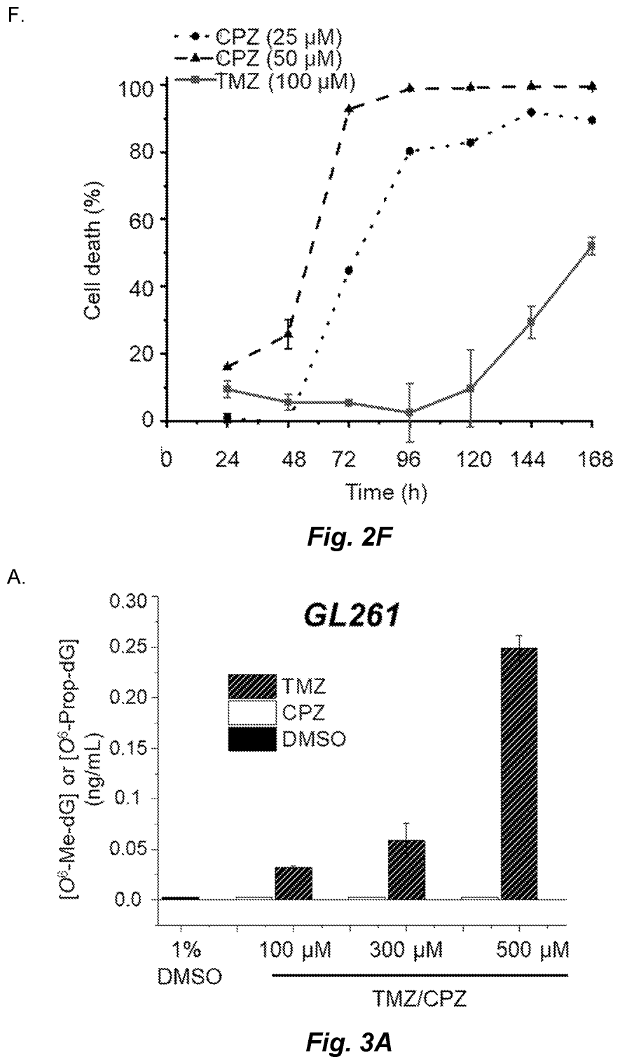

Mode of Action Studies of CPZ. CPZ exhibits superior anticancer efficacy in each cell line tested compared to its precursor 10, and most importantly, it retains activity in MGMT-expressing cell lines with an average IC₅₀ value of 16 μM in MGMT (−) cell lines versus 22 μM in MGMT (+) cell lines (FIG. 2A). Beyond MGMT, loss of a functional MMR system is another primary resistance mechanism to TMZ, both in cell culture and in human GBM patients. Therefore, GBM tumors that express MGMT and/or have MMR deficiencies suppress the activity of TMZ. These clinical scenarios were recapitulated in culture by employing cell lines with different MGMT/MMR status; when possible, cell lines derived from human brain tumors were used (FIG. 2B-2E). As shown in FIG. 2B-2E, the MGMT (−)/MMR (+) U87 cell line was responsive to TMZ (IC₅₀˜50 μM), while the MGMT (+)/MMR (+) T98G cell line was not (IC₅₀>550 μM), consistent with clinical results; CPZ was effective against both of these cell lines (FIG. 2B-2E).

To assess the necessity of the MMR system, the activity of TMZ and CPZ was evaluated against MGMT (−)/MMR (−) HCT116 cells, which have a mutated MLH1 gene. As expected, these cells were insensitive to TMZ with an IC₅₀ value >800 μM (FIG. 2B-2E). Importantly, CPZ was still able to potently elicit cell death with an IC₅₀ value of 14 μM. Perhaps most striking, CPZ has efficacy in two different MGMT (+)/MMR (−) cell lines. The D341 Med medulloblastoma cell line is highly resistant to TMZ (IC₅₀=460 μM), while CPZ is able to induce cell death with low micromolar potency (IC₅₀=8 μM, FIG. 2B-2E). Similarly, the RKO colon cancer cell line, also MGMT (+)/MMR (−) and exceptionally resistant to TMZ (IC₅₀>1000 μM), is potently killed by CPZ (IC₅₀=10 μM). The convincing cell death elicited by CPZ in these cell lines suggests complete avoidance of the two primary resistance mechanisms to TMZ.

The striking anticancer activity even in an MMR-deficient cell line indicates that CPZ may be operating, at least in part, through a unique mode of cell death. Indeed, when the timing of cell death was studied, CPZ was close to reaching its peak threshold of cell death within 72 hours in several cancer cell lines (FIG. 2F). In contrast, TMZ (at 100 μM) takes between 5-10 days to induce cell death in culture (FIG. 2F), known to require >2 futile cycles of DNA replication before apoptosis is initiated.

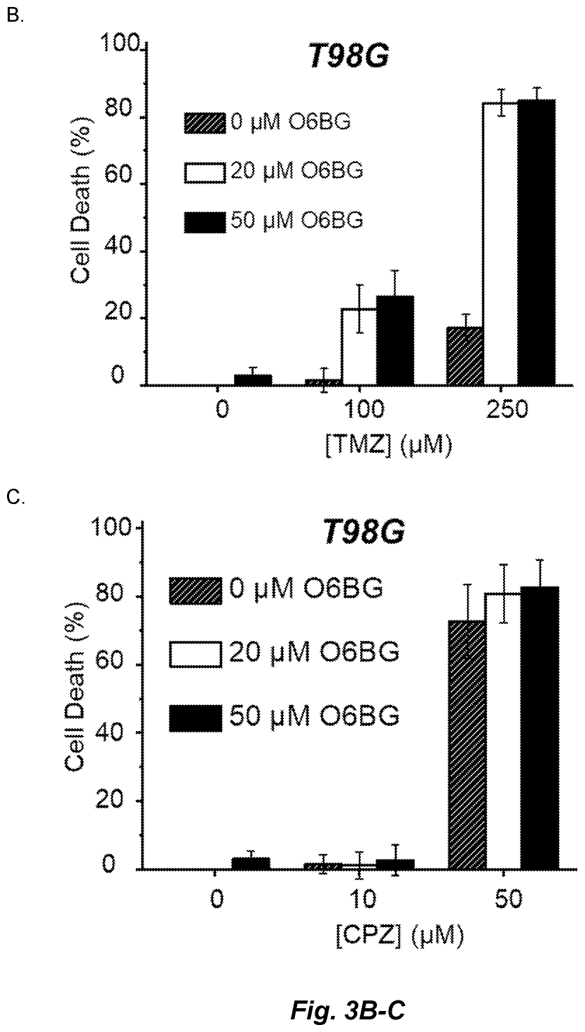

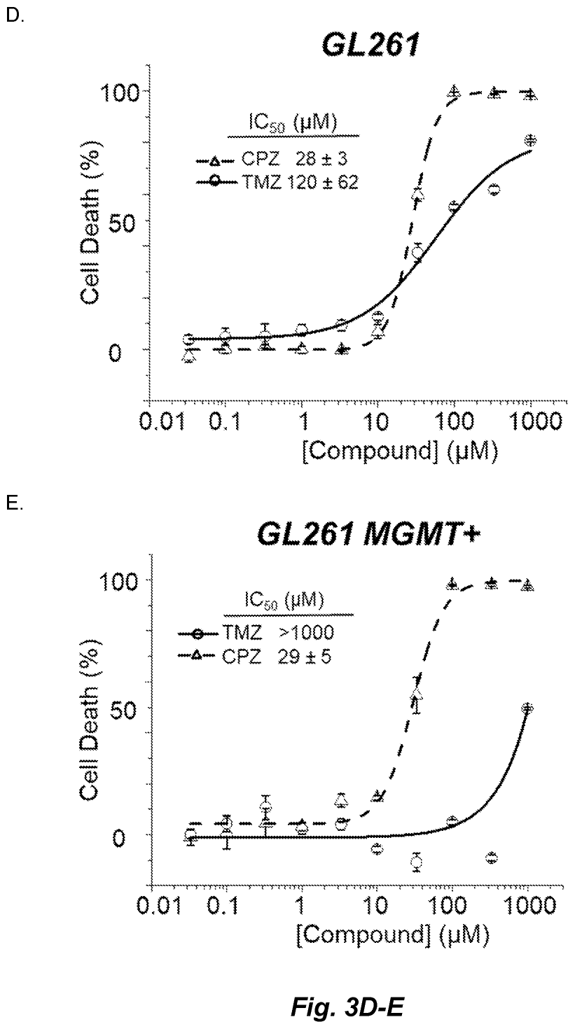

Importantly, because CPZ was designed to release a different active species than TMZ, the alkylation pattern in a cellular context is likely different. Therefore, to assess if alkylation still occurs with CPZ, a DNA alkylation assay was performed. GL261 cells were treated with 100, 300, or 500 μM TMZ or CPZ for 8 hours after which the cells were harvested and genomic DNA was extracted and subsequently hydrolyzed to constituent deoxyribonucleosides. LC-MS/MS analysis was employed to quantify the amount of O⁶-methyl-2′-deoxyguanosine (O⁶-Me-dG) or O⁶-propargylated-2′-O⁶-deoxyguanosine (O⁶-Prop-dG) in each sample. A clear dose-dependent increase in the concentration of O⁶-Me-dG was observed in the GL261 cell line after treatment with TMZ. Strikingly, there was no detection of O⁶-Prop-dG adducts for CPZ (FIG. 3A). To evaluate if this result was unique to GL261, O⁶-dG alkylation adducts of TMZ and CPZ were quantified in two other MGMT-negative GBM cell lines, A172 and D54. Consistent with the results observed in GL261 cells, TMZ-treated cells showed a dose-dependent increase in methyl adducts whereas CPZ-treated cells did not harbor measurable levels of the propargyl adduct. The undetectable concentrations of O⁶-Prop-dG adducts versus the clear detection and dose-dependent increase of O⁶-Me-dG adducts suggests a different alkylation pattern for CPZ or an alternate fate for the propargyl active species.

To probe if CPZ releases the propargyl diazonium ion, CPZ was hydrolyzed under various alkaline conditions in an attempt to recover the corresponding 4-chloro-5-aminoimidazole byproduct. Recovery of this species would permit the reasonable assumption that the putative propargyl diazonium ion was released. However, attempts to identify 4-chloro-5-aminoimidazole were not fruitful, likely a consequence of its instability, unsurprising given the absence of this compound in the literature. Instead, when 10, which bears the same propargyl N3 group, was hydrolyzed under alkaline aqueous conditions, the presence of 5-amino-1H-imidazole-4-carboxamide (AIC) was detected via LC/MS and aligned with both a synthetic standard and AIC recovered from degraded TMZ. This result suggests that propargyl-bearing imidazotetrazines can hydrolyze in a manner akin to TMZ.

To further explore the ability of CPZ to evade MGMT-mediated resistance, MGMT-expressing T98G cells were pretreated with MGMT inhibitor O6BG followed by treatment with TMZ or CPZ. A dramatic potentiation of cell death was observed for TMZ relative to non-pretreated cells confirming that MGMT is responsible for the ineffectiveness of TMZ in this cell line (FIG. 3B, 3C). No such enhancement in activity was observed for CPZ (FIG. 3B, 3C), suggesting that MGMT expression does not confer resistance to CPZ in these cells. This cell death potentiation by TMZ upon treatment with an MGMT inhibitor was also observed in MGMT-expressing cell lines U118MG and RKO, whereas the efficacy of CPZ was again unaffected. For MGMT (−)/MMR (−) HCT116 cells, negligible potentiation of TMZ cytotoxicity was observed upon pretreatment with O6BG, confirming that loss of MMR capacity, not MGMT expression, was responsible for resistance to TMZ in HCT116 cells. Additionally, human MGMT was knocked into the MGMT-negative mouse glioma cell line, GL261, establishing the novel cell line GL261 MGMT+. The IC₅₀ values for TMZ and CPZ in the parental cell line are 120 μM and 28 μM, respectively (FIG. 3D). The isogenic knock-in abolished all TMZ activity (IC₅₀>1000 μM), whereas CPZ retains its efficacy (IC₅₀=29 μM) (FIG. 3E). Similarly, pre-treatment of the knock-in cells with MGMT inhibitor lomeguatrib shows potentiation of cell death for TMZ but not for CPZ.

Next, we measured the mutagenicity of CPZ versus TMZ. TMZ and other DNA methylating chemotherapies are known mutagens, inducing G:C→A:T mutations due to erroneous recognition of O⁶-methylguanine. This was reflected by a positive result in the Ames test; in the S. typhimurium TA100 strain, 35/96 colonies were mutated 5 days after treatment with 30 μM of TMZ whereas no revertant colonies were observed when treated with the same concentration of CPZ. CPZ and TMZ (at 30 μM) were also assessed in the E. coli WP2 uvrA pKM101 strain, which is considered one of the more sensitive Ames tester strains available. Indeed, TMZ was found to be highly mutagenic in this strain, with 95/96 revertant colonies. In contrast, CPZ again showed no revertant colonies, indicating that at this concentration and in this strain, CPZ is non-mutagenic.

To further probe the molecular basis of CPZ-induced cell death, CPZ was evaluated in a biochemical DNA alkylation assay that has been used to elucidate the mechanism of DNA damaging small molecules. In the experiment, purified linearized DNA was incubated with compound for 15 hours at 37° C. The DNA was subsequently denatured and the sample was eluted on a 1% agarose gel. Interstrand DNA cross-linkers like cisplatin prevent the full denaturation of double-stranded DNA, while DNA alkylating agents like MMS or TMZ cause DNA streaking due to shorter DNA fragments that are formed upon alkaline denaturation of abundantly alkylated DNA. Interestingly, CPZ exhibited clear evidence of both DNA cross-linking and alkylation. The cross-linking and alkylation are dose-dependent and occur quickly, observable within minutes after dosing.

To probe the effect of the alkylating substituent on DNA alkylation and cross-linking in this assay, 3, 9, and 2-butyne imidazotetrazine 19 were also evaluated. No evidence of DNA alkylation was observed for 3 or 9, perhaps due to more unstable diazonium ion species. Trace DNA alkylation was observed after treatment with 500 μM of 19, but evidence of cross-linking was definitively absent suggesting cross-linking with CPZ was a product of S_{N}2′ reactivity via attack at the terminal carbon of the alkyne. Finally, to ensure that the cross-linking phenotype exhibited by CPZ was not a product of the assay conditions, unbuffered water (pH 7) and less alkaline denaturation conditions (0.2% NaOH vs. 1% NaOH) were implemented. Both conditions revealed results similar to that observed previously, suggesting that the cross-linking activity of CPZ is independent of the assay conditions. These results show that in a non-cellular context CPZ can alkylate DNA (in addition to cross-linking); taken together with undetectable level of the O⁶-dG adducts in cells treated with CPZ, further studies are warranted to elucidate the major adducts formed and their contribution to the cytotoxicity of CPZ.

Two putative interstrand cross-linking mechanisms can be envisaged for CPZ, both a result of S_{N}2′ attack on the intermediate propargyl diazonium species 20 (Scheme 2). The first would generate DNA cross-links via an allenyl-DNA intermediate formed after S_{N}2′ attack on 20 by a nucleophilic site on DNA (e.g. N⁷ guanine). A second plausible mechanism involves an initial S_{N}2′ addition by water, forming 21 (Scheme 2). Compound 21 can readily tautomerize to 22 (acrolein), which is a known DNA interstrand cross-linker via a multistep mechanism; indeed, subjecting reagent-grade acrolein to the DNA alkylation assay under identical conditions led to interstrand cross-linking but no evidence of alkylation. A trapping experiment was performed with acrolein to assess the feasibility of mechanism 2. Upon incubation of acrolein with 2′-deoxyguanosine in PBS, a new peak formed by LC-MS with a mass matching the expected adduct (M+1 m/z=324; loss of deoxyribose m/z=208). However, incubation of CPZ with 2′-deoxyguanosine under identical conditions did not produce any trace of the same adduct. This result preliminarily suggests that significant amounts acrolein are not generated when the propargyl diazonium ion is released, and thus favors mechanism 1 (Scheme 2) as the source of CPZ's crosslinking.

Scheme 2. Two potential mechanisms for DNA cross-link formation from propargyl diazonium ion. Mechanism 1 proceeds through an allenyl DNA adduct (A). Mechanism 2 evokes acrolein as the active cross-linking agent (B).

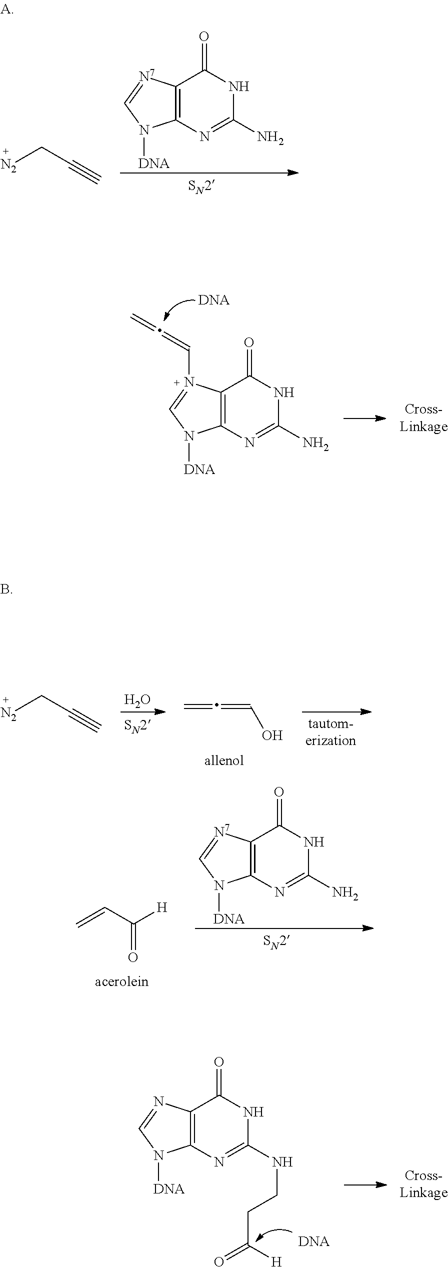

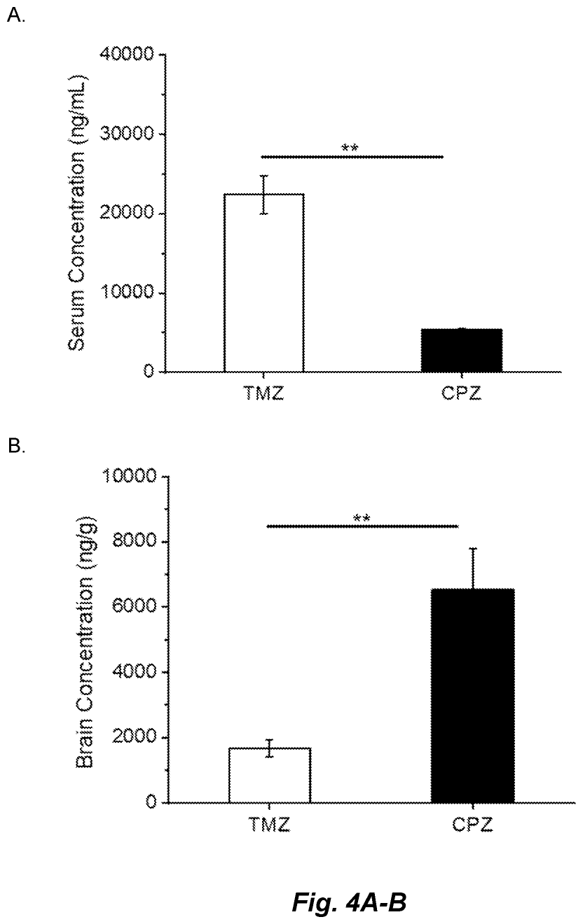

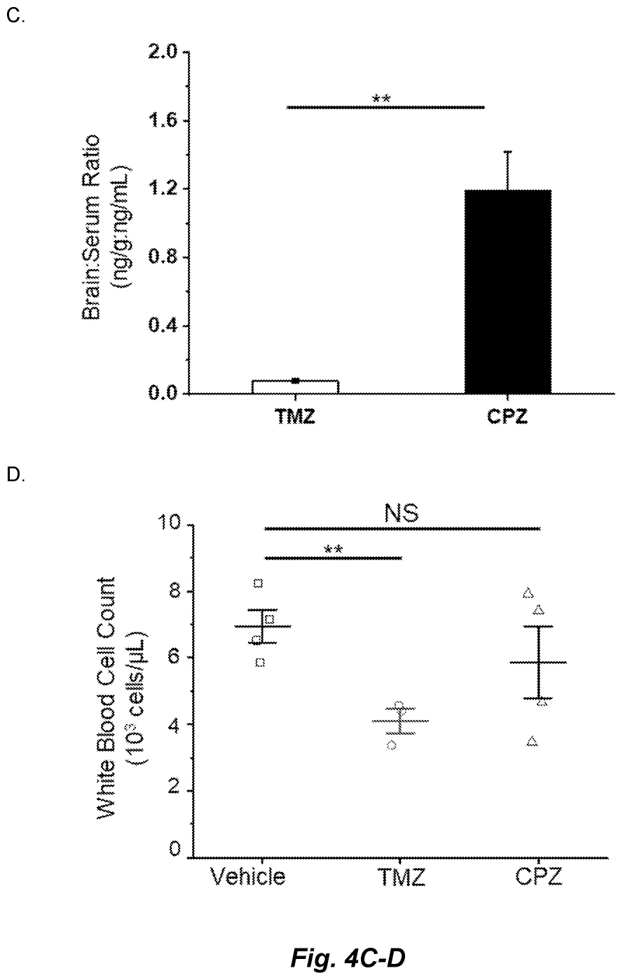

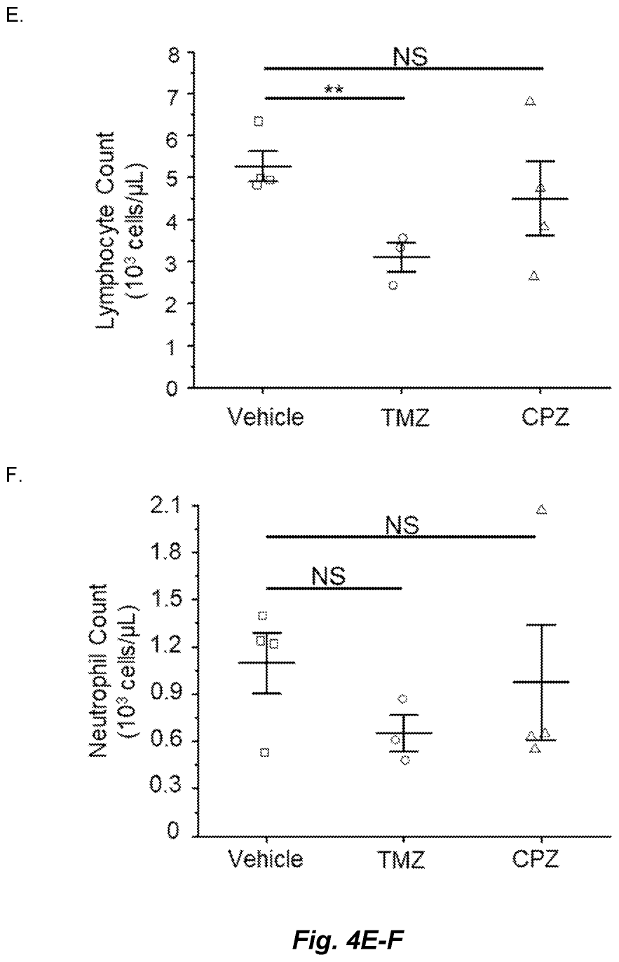

Biodistribution and Toxicity Studies. In humans, the cerebral spinal fluid concentration of TMZ averages about 20% of the concentration in the plasma. Accumulating even more compound in the brain may be a beneficial strategy to increase imidazotetrazine efficacy against CNS tumors. Of equal importance, targeting more drug to the brain and less to the plasma may offset some of the expected hematological toxicity of an MGMT-independent imidazotetrazine. To investigate the BBB penetrance of TMZ and CPZ, mice were administered 25 mg/kg intravenously then sacrificed after 15 minutes. Brain tissue and blood were collected from each mouse and immediately acidified to prevent prodrug degradation. The concentration of each compound in the serum (FIG. 4A) and brain (FIG. 4B) were quantified by LC-MS/MS. TMZ had a brain:serum ratio of 0.08±0.01 ng/g:ng/mL (FIG. 4C), commensurate with other TMZ biodistribution studies in murine systems. Compound 10 had a near-equivalent ratio of 0.06±0.01 ng/g:ng/mL indicating that swapping a methyl group at N3 for a propargyl group had a negligible effect on BBB penetrance as suggested by cLogBB and CNS MPO scores (Table 2). However, CPZ demonstrated a >10-fold increase in brain distribution relative to TMZ with a ratio of 1.2±0.2 ng/g:ng/mL (FIG. 4C).

Arguably the most important question when designing an alkylating agent able to evade MGMT-mediated resistance is the resultant effect on hematological toxicity. MGMT operates as a systemic protectant against alkylating xenobiotics. Consequently, its inhibition leads to drastically enhanced sensitivity to the toxic effects of alkylating chemotherapy. Therefore, an MGMT-independent alkylating agent like CPZ could render off-target cells vulnerable to irreparable DNA alkylation and result in a similar degree of toxicity. We hypothesized that the increased BBB permeability of CPZ would divert enough of the drug to the brain such that it could be tolerated at a therapeutically useful dose. The hematological effects of a single dose of TMZ or CPZ were compared head-to-head in vivo. Mice were treated with 125 mg/kg TMZ or CPZ intravenously; this dose was selected because 125 mg/kg TMZ is known to induce nonlethal toxicity in mice. After 7 days, the mice were sacrificed and complete blood counts were obtained. Expectedly, 125 mg/kg of TMZ led to depletions in white blood cells, lymphocytes, and neutrophils relative to vehicle-treated mice, representing drug-induced myelosuppression (FIG. 4D-4F). CPZ-treated mice exhibited total white blood cell, lymphocyte, and neutrophil counts that were similar to TMZ. Notably, CPZ did not give rise to other hematological symptoms such as thrombocytopenia. This could suggest that more BBB penetrant imidazotetrazines mitigate drug-induced hematological toxicity; however, it is also recognized that the exact mechanism of cell death for CPZ, including the biological processing and alkylation pattern of the propargyl moiety, is not yet clear. Additionally, the maximum tolerated dose (MTD) of CPZ was determined to be 66 mg/kg when dosed intraperitoneally (IP), once-per-day over five days. This matches the reported MTD of TMZ at the same schedule (˜66 mg/kg).

### DISCUSSION

TMZ has been a mainstay as the standard-of-care therapy for GBM patients since its approval in 2005, and remains frontline therapy for other brain cancers such as oligodendrogliomas and diffuse astrocytic gliomas. In patients whose tumors do not express MGMT and have functioning MMR, TMZ extends GBM patient survival by nearly one year compared to patients receiving RT only. Unfortunately, GBMs that express MGMT and/or have reduced MMR capacity are not sensitive to TMZ, and this describes a majority of the GBM patient population both newly diagnosed and recurrent. Therefore, a variant of TMZ that is able to extend survival benefits to GBM patients with MGMT-expressing and MMR-deficient tumors would have a transformative clinical impact. The 30+ year absence of an approved imidazotetrazine that appends a group apart from methyl underscores the immensity of the challenges faced when modifying imidazotetrazines: synthetic accessibility, rate of hydrolysis, BBB permeability, toxicity profile, etc., and suggests that simply substituting the methyl group at N3 with another functional group is unlikely to yield MGMT-independent anticancer activity that properly balances all other factors.

The dual-substituted compound CPZ displays anticancer activity irrespective of MGMT expression and MMR status. Though MGMT promoter methylation is predictive of tumoral response to TMZ, the biomarker is not widely actionable because of extensive intertumoral heterogeneity and the lack of other therapeutic options. As a result, all GBM patients are treated with TMZ despite its ineffectiveness in most of these patients. The importance of MMR for patients receiving TMZ extends beyond futile cycling of O⁶-methylguanine. TMZ chemotherapy in MGMT-negative GBM induces a strong selective pressure to mutate or downregulate the MMR pathway, usually through MSH6, MSH2, MLH1, or PMS2. Recent studies have shown that in addition to conferring resistance to TMZ, loss of MMR function unleashes the mutagenic potential of TMZ as unrepaired O⁶-methylguanine adducts lead to widespread G:C→A:T transition mutations, more malignant hypermethylated tumors, and a positive result in the Ames test. Contrastingly, CPZ leads to direct lethality in cancer cells and appears to avoid a lesion-tolerant, hypermutated phenotype as suggested by negative results in two Ames tests, including in a strain more sensitive to mutation. However, it should be noted that these two strains are sensitive to mutation via direct base substitution, and use of other strains to interrogate other possible forms of mutagenicity (e.g. frame shift, cross-linking, oxidative damage, etc) will be critical to determine the full mutagenic profile of CPZ. Killing GBM cells through a less mutagenic lesion could mitigate or delay tumor recurrence and/or progression by preventing the acquisition of additional driver mutations.

In addition to alkylating DNA in biochemical experiments, CPZ is also capable of cross-linking DNA, and a cross-linking imidazotetrazine immediately invites comparison to first-generation imidazotetrazine, mitozolomide (MTZ). Despite showing curative anticancer activity in murine models, MTZ met a swift end in the clinic due to extreme myelosuppressive effects that were attributed to DNA cross-linking. Not to be understated, the single dose MTD of MTZ in mice is 37.5 mg/kg, whereas CPZ can readily be dosed 66 mg/kg once-per-day over 5 days. Careful consideration of the active cross-linking species reveals key differences between CPZ and MTZ. Both the anticancer activity and toxicity of MTZ has been attributed to interstrand DNA cross-links as only ˜20 per cell are required for lethality (versus >6500 O⁶-methylguanine lesions). Indeed, a comparison of the cytotoxicity of N3-ethyl imidazotetrazine (2, U87 IC₅₀=470 μM) and N3-chloroethyl imidazotetrazine (MTZ, U87 IC₅₀=30 μM), which differ only by a single chlorine atom, suggests that MTZ derives nearly all of its anticancer efficacy in culture from its ability to cross-link DNA.

In contrast to MTZ, the DNA interstrand cross-links imparted by CPZ are hypothesized to arise from a side reaction of the propargyl diazonium ion (S_{N}2′ vs. S_{N}2). Therefore, the cytotoxicity of CPZ appears not to be primarily driven by the interstrand cross-links. The contribution of CPZ cross-links to cell death can be estimated by the difference in activity between compounds 10 and 19. The slight difference in cytotoxicity (IC₅₀=29 μM vs. 37 μM in U87 and 77 μM vs. 110 μM in T98G for 10 and 19, respectively) observed may be due to the ability of 10 to cross-link where 19 cannot (S_{N}2′ pathway is blocked). The retention of cytotoxicity by 19 serves as further evidence that the anticancer activity of N3-propargyl imidazotetrazines is likely driven by propargylic monoadducts, although at this time the exact pattern of alkylation is not known.

The observations reported herein challenge a major assumption in the imidazotetrazine literature that N3 derivatives of TMZ operate analogous to TMZ, through formulation of O⁶-dG alkyl lesions; to date, the effect of propargyl and other diazonium ions on DNA has not been explored in depth. Our cell-free DNA alkylation experiments suggest that DNA alkylation is occurring, but cell-based LC-MS/MS quantitation indicates that O⁶-Prop-dG adducts are not formed at a detectable level, and thus such lesions are not likely to be the driver of cell death, in stark contrast with TMZ and the analogous methyl lesion. Instead, propargyl diazonium ions delivered by CPZ induce rapid cytotoxicity across cell lines. Future studies are required to identify the exact DNA alkylation pattern and to trace the contribution of the major adduct(s) to DNA and cancer cell death.

The compounds described herein (CPZ and others) now provide the appropriate tools to test the hypothesis that an MGMT-evading imidazotetrazine can be safe and efficacious in intracranial in vivo models of GBM. In addition, the GL261/GL261 MGMT+ isogenic cell line pair reported here will be an extremely useful tool for in vivo studies, since a syngeneic model provides the opportunity to assess compound activity in the presence of an intact immune system while recapitulating the chemoresistance exerted by MGMT. While CPZ and other compounds reported herein provide reason for optimism, it is notoriously difficult to out-perform TMZ in intracranial models; almost exclusively, imidazotetrazines that show promise in cell culture fail in in vivo models, with only one compound showing a meaningful difference compared to TMZ.

In summary, we have identified a novel imidazotetrazine that circumvents the two primary resistance mechanisms to TMZ, expression of MGMT and loss of MMR function. Despite its drawbacks, TMZ remains the backbone of glioblastoma treatment. However, CPZ and TABZ are exciting alternatives that may improve the clinical situation of patients that cannot be successfully treated beyond surgery and RT. The assessment of these compounds head-to-head in intracranial tumors will be reported in due course.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.50% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-tumor agents and have higher potency and/or reduced toxicity as compared to TMZ. Preferably, compounds of the invention are more potent and less toxic than TMZ, and/or avoid a potential site of catabolic metabolism encountered with TMZ, i.e., have a different metabolic profile than TMZ.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, colon cancer, breast cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known. In addition, ability of a compound to treat cancer may be determined using the procedures as described herein.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Examples

### Example 1. Materials and Methods

Chemistry. Chemical reagents were purchased from commercial sources and used without further purification. Flash chromatography was performed using silica gel (230-400 mesh). Anhydrous solvents were dried after being passed through columns packed with activated alumina under positive pressure of nitrogen. Unless otherwise noted, all reactions were carried out in oven-dried glassware with magnetic stirring under nitrogen atmosphere. 1H and 13C NMR spectra were recorded on Bruker 500 (500 MHz, 1H; 125 MHz, 13C) or Varian Unity Inova 500 (500 MHz, 1H) MHz spectrometers. Spectra are referenced to residual chloroform (δ=7.26 ppm, 1H; 77.16 ppm, 13C) or dimethyl sulfoxide (δ=2.50 ppm, 1H; 39.52 ppm, 13C). Multiplicities are indicated by s (singlet), d (doublet), t (triplet), q (quartet), m (multiplet), and br (broad). Coupling constants J are reported in Hertz (Hz). High resolution mass spectrometry (HRMS) was performed on a Waters Q-T of Ultima or Waters Synapt G2-Si instrument with electrospray ionization (ESI) or electron impact ionization (EI).

Cell Culture and Reagents. All cell lines were grown in a 37° C., 5% CO2, humidified environment, in media containing 1% penicillin/streptomycin. Stable cell line generation of GL261 MGMT+ was purchased from Creative Biogene. Cell culture conditions are as follows: cell lines U87, T98G, and RKO were grown in EMEM with 10% FBS. D54, GL261, GL261 MGMT+, and U118MG cell lines were grown in DMEM with 10% FBS. HCT116 cells were grown in RMPI with 10% FBS. D341 Med cells were grown in EMEM with 20% FBS. Temozolomide (TMZ) was purchased from AK Scientific. TMZ analogues were synthesized as described below. Compounds were dissolved in DMSO (1% final concentration, Fisher Chemical) for cell culture studies.

Bacterial Strains. S. typhimurium 14028 was obtained from the American Type Culture Collection (ATCC).

Antibodies. Antibodies used herein: MGMT: CST-2739, Anti-rabbit IgG HRP-linked: CST-7074, GADPH: CST-2118.

### Procedures for Biological Assays.

Cell Viability Assays. Cells were harvested, seeded in a 96-well plate and allowed to adhere. After three hours, compound was added to each well in DMSO (1% final concentration). Cells were incubated for seven days (unless otherwise noted) before viability was assessed by the Alamar Blue assay. Raptinal (20 μM) was used as a dead control.

Hydrolytic Stability Studies. Compound (100 μM) was incubated at 37° C. in PBS at pH 7.4 (unless otherwise noted). At the indicated time point, an aliquot was collected and analyzed by HPLC. Compound peaks were integrated and compared to the integration at to calculate the percentage of prodrug remaining.

Killing Kinetics Study. U87 cells were seeded in seven 96 well plates starting at 19,200 cells/well (for 24 hour time point) and serially diluted by two-fold for each subsequent 24 hour time point. At each time point, cell viability was assessed via Alamar Blue assay.

Quantitation of O⁶-guanine DNA Adducts in Cells. GL261, A172, or D54 cells were plated at 1×10⁶ c/w in a 6-well plate before they were treated with compound at the indicated concentration (1% final concentration DMSO). After incubation for the indicated time, the cells were harvested and pelleted. Genomic DNA was extracted using the DNeasy Blood & Tissue Kit (Qiagen, ID: 69504). DNA was then precipitated using the following procedure: 1/10 v/v 3 μM sodium acetate (pH 5.2) and 2.5× v/v ethanol was added to each sample which was then kept at −80° C. for 1 h. The mixture was centrifuged at max at 4° C. for 30 min and decanted to afford a pellet of DNA, which was re-suspended in ddH2O containing 10 mM tris base (pH 7.5) and 1 mM EDTA. The concentration of DNA in each sample was quantified by measuring absorbance on a NanoDrop 2000 UV-Vis Spectrophotometer (Thermo Fisher). DNA (10 μg) from each sample was added to DNA hydrolysis buffer and incubated at 37° C. for 6 h. Hydrolyzed samples were then submitted for LC-MS/MS quantitation along with a synthetic standard. Samples were analyzed with a 5500 QTRAP LC/MS/MS system (AB Sciex) with a 1200 series HPLC system (Agilent).

Immunoblotting Western Blot Procedure. Cells were lysed using RIPA buffer containing protease inhibitor cocktail (Calbiochem). Protein concentrations were determined using the BCA assay (Pierce). Lysates containing 10 μg of protein were loaded onto 4-20% gradient gels (BioRad), and SDSPAGE was run. For GL261 MGMT+, the lysate was diluted ×20 for optimal visualization of MGMT band. Proteins were then transferred onto membrane (PDVF Millipore) for Western Blot analysis. Blots were blocked with BSA solution (2 g in 40 mL TBST) for one hour followed by primary antibody addition (1:1000) and incubation overnight. Following overnight incubation, blots were washed with TBST, and incubated with HRP-linked secondary antibody for 1 hour in TBST. Blots were washed, then imaged with ChemiDoc after incubation with SuperSignal West Pico Solution following manufacturer's procedures.

Antimicrobial Susceptibility Tests. Susceptibility testing was performed in biological triplicate, using the micro-dilution broth method as outlined by the Clinical and Laboratory Standards Institute. Bacteria were cultured with cation-adjusted Meuller-Hinton broth (Sigma-Aldrich; catalogue number: 90922) media in round-bottom 96-well plates (Corning; catalogue number: 3788).

Ames Test. Compounds were assessed at the indicated concentrations in the TA100 tester strain using the Muta-ChromoPlate 96-well microplate version of the Ames test (Environmental Bio-Detection Products, Prod. No. 5051) according to manufacturer's instructions. In brief, TA100 S. typhimurium bacteria were grown overnight and plated with compound, growth medium, and indicator (no S9 activation) in 96 well plates. Bacteria were incubated for 5 days before the number of wells per 96 well plate containing revertant colonies were identified colorimetrically.

CPZ and TABZ at 30 μM, in these strains, were non-mutagenic. TMZ was mutagenic in both strains.

DNA Cross-Linking/Alkylation Assay. Procedure was adapted from Healy et al. (61) In brief, pBR322 plasmid DNA was linearized with EcoRI (New England BioLabs) in NEB EcoRI buffer (New England BioLabs) according to the manufacturer's instructions. The cut plasmid DNA was purified using a PCR cleanup kit (QIAquick PCR Purification Kit, Qiagen) and eluted into DNA buffer (10 mM Tris Cl, pH 8.5). The concentration of linearized DNA was quantified by measuring absorbance on a NanoDrop 2000 UV-Vis Spectrophotometer (Thermo Fisher). For each reaction, compound (in DMSO, 5% v/v) was added to DNA (150 ng) in DNA buffer (total volume 10 L) and incubated at 37° C. for the indicated time. Upon completion, 30 L DNA denaturation buffer (60 mg/mL sucrose, 10 mg/mL NaOH, and 0.4 mg/mL bromophenol blue in water) was added to each sample (the same buffer without NaOH was added to non-denatured controls). Samples were vortexed and denatured for 15 min at 4° C. before they were loaded onto a 1% neutral agarose gel (containing ethidium bromide) and run in TBE buffer (containing ethidium bromide) at 124 V for 1 hour. Gels were imaged using a Gel Dox XR+ system (Bio-Rad).

Acrolein TrappingAssay. Acrolein (1 mmol) was incubated with 2′-deoxyguanosine (0.09 mmol) at 37° C. in 10×PBS (10 mL, pH=7) overnight. The reaction mixture was directly injected on LC/MS without prior purification to visualize acrolein adducts. 2′-deoxyguanosine (0.09 mmol)/temozolomide (1 mmol) and 2′-deoxyguanosine (0.09 mmol)/CPZ (1 mmol) mixtures were separately incubated in 10×PBS (10 mL, pH=7) overnight. These reaction mixtures were directly injected on LC/MS without prior purification to look for the same adduct generated from the acrolein adduct standard as described.

Blood-Brain Barrier Permeability. All experimental procedures were reviewed and approved by the University of Illinois Institutional Animal Care and Use Committee. CD-1 IGS mice were administered compound in 5% DMSO, 10% Tween-20, and 85% SPE-PCD in sterile water (30% w/v) at 25 mg/kg via lateral tail vein injection. Fifteen minutes post injection, mice were sacrificed and blood was collected by lacerating the right auricle with iris scissors. An 18 gauge angiocatheter was inserted through the left ventricle, and all residual circulatory volume was removed by perfusing 0.9% saline solution via an analog peristaltic pump. Blood samples were immediately centrifuged at 13,000 rcf for five minutes and the supernatant collected and acidified with 8.5% aqueous H₃PO₄. Brains were harvested from the cranial vault, acidified with 0.3% aqueous H₃PO₄ and flash frozen. Homogenized brain samples were centrifuged twice at 13,000 rcf for ten minutes and supernatant and tissue debris were separated. The resultant supernatant was analyzed, along with plasma, by LC-MS/MS to determine compound concentrations.

Assessment of Hematological Toxicity. Male CD-1 IGS mice (n≥3 mice/group) were administered a single dose of 125 mg/kg compound intravenously. Imidazotetrazines were formulated with DMSO, Tween-20, and 30% (w/v) SBE-PCD in sterile water immediately prior to injection. Seven days post-treatment, mice were humanely sacrificed and whole blood was collected for assessment of total white blood cells, lymphocytes, neutrophils, platelets, and red blood cells.

Assessment of Maximum Tolerated Dose (MTD). Female C57BL/6 mice were administered compound via intraperitoneal injection in 35% PEG400, 25% propylene glycol, 6% Tween-80, and 34% sterile water (10 mL/kg injection volume) at 66 mg/kg once per day over 5 days. Mice were monitored for weight loss and other signs of toxicity; no significant weight loss or signs of toxicity were observed for this dosing schedule.

CPZ insufficient solubility for IV administration. TABZ is able to be dosed ×2/day over 5 days at 66 mg/kg.

### Example 2. Characterization and Preparation of N3 Analogs (Schemes 3 and 4)

TABLE 4
Activities (micromolar) of Imidazotetrazines.

| | Cell line | TMZ | CPZ | TABZ | 16 | 17 | 20 |
|---|---|---|---|---|---|---|---|
| MGMT (−) | U87 | 51 ± 8 | 16 ± 6 | 16 ± 2 | 40 ± 20 | 20 ± 5 | 50 ± 6 |
| MMR (+) | GL261 | 120 ± 62 | 28 ± 3 | 35 ± 2 | ND | ND | 86 ± 4 |
| MGMT (+) | GL261 | >1000 | 29 ± 5 | 18 ± 1 | ND | ND | 70 ± 2 |
| MMR (+) | MGMT+ | | | | | | |
| | U118MG | 322 ± 7 | 17 ± 1 | 24 ± 5 | ND | ND | 70 ± 7 |
| | T98G | 570 ± 20 | 27 ± 6 | ND | 87 ± 7 | 40 ± 5 | ND |
| MGMT (−) | HCT116 | 840 ± 30 | 14 ± 16 | 6 ± 0.42 | ND | ND | ND |
| MMR (−) | | | | | | | |
| | RKO | >1000 | 10 ± 1 | 9 ± 1 | ND | ND | ND |

Plated 1500 c/w for GL261; 600 c/w for U87; 600 c/w for U118MG; 1500 c/w for GL261 MGMT+ Incubated ovn and added 1 μL of compound in DMSO the next day. Incubated over 7 days; added Alamar Blue to assess cell viability and used 10 μM Raptinal as dead control. Error is SEM, n=3.

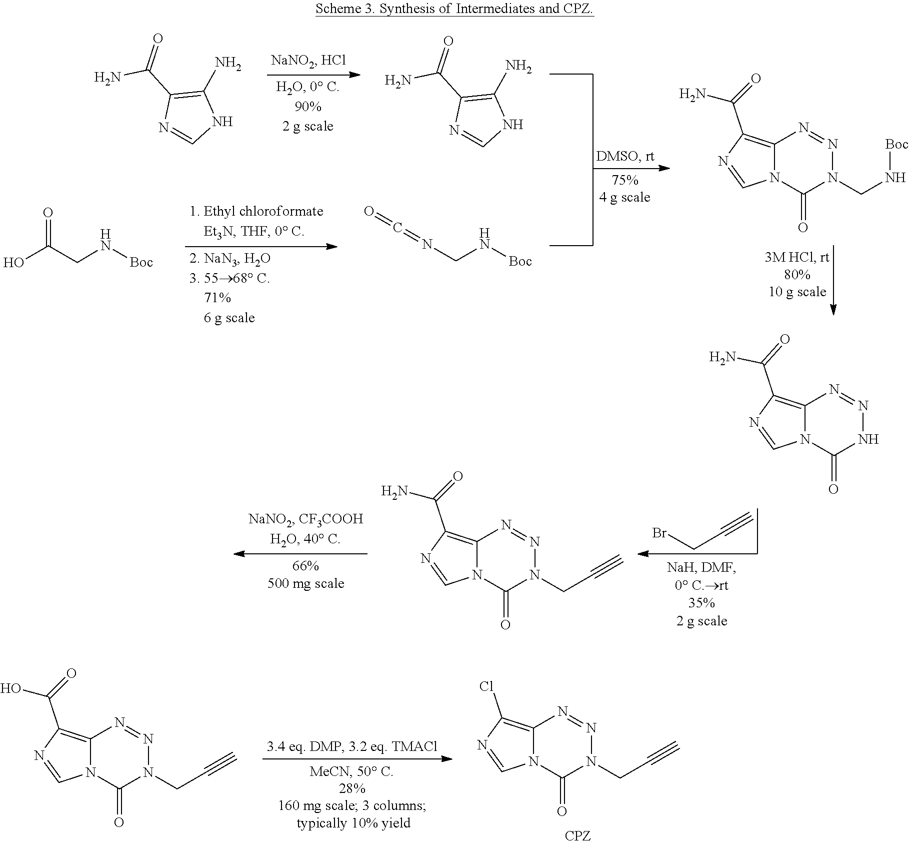

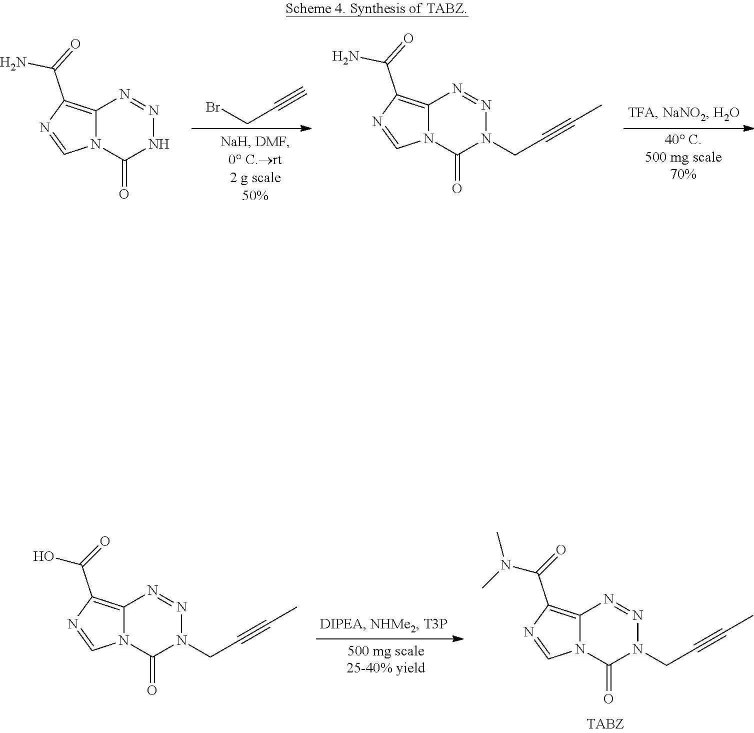

Note: Experimental information has been previously reported for 13, 14 (Molecules 2010, 15, 9427) and 15 (ACS Chem Biol 2018, 13, 3206) (Scheme 1).

### General Procedure A.

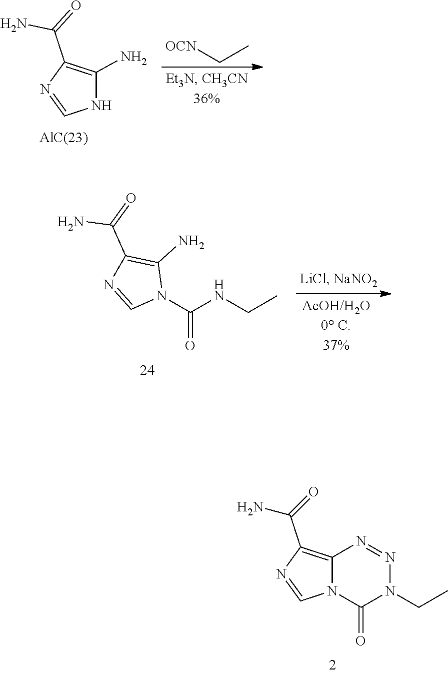

To an oven dried 25 mL round bottom flask, 4-aminoimidazole-5-carboxamide (23, 505.0 mg, 3.1 mmol, 1 eq.) was added and suspended in dry acetonitrile (7.0 mL, 0.44 M). Anhydrous triethylamine (0.94 mL, 6.7 mmol, 2.2 eq.) was added and the flask was flushed with nitrogen before ethyl isocyanate (0.27 mL, 3.4 mmol, 1.1 eq.) was added slowly. The reaction was stirred overnight under nitrogen. Upon completion, the reaction mixture was filtered and washed with water and diethyl ether to afford intermediate 24 as a white solid (222.6 mg, 1.13 mmol, 36% yield).

To a 25 mL round bottom flask, LiCl (1.19 g, 28.1 mmol, 31.6 eq.), water (2.0 mL, 0.45 M), and acetic acid (0.17 mL, 5.2 M) were added. After 30 min, compound 24 (175.1 mg, 0.89 mmol, 1 eq.) was added. After another 30 min, the reaction was cooled to 0° C. and sodium nitrite (85.9 mg, 1.24 mmol, 1.4 eq.) was added slowly as a solution in a minimal amount of water. The reaction was stirred at 0° C. for 1 hour and then at room temperature for 4 hours. The resultant mixture was extracted ×6 with dichloromethane, washed xl with saturated sodium bicarbonate before it was dried over sodium sulfate and concentrated. The crude solid was purified by column chromatography (100% ethyl acetate) to afford 2 as a white solid (68.8 mg, 0.33 mmol, 37% yield).

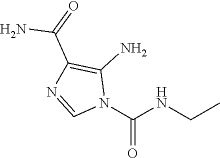

5-amino-N¹-ethyl-1H-imidazole-1,4-dicarboxamide (24). ¹H NMR (500 MHz, d-DMSO) S 8.47 (s, 1H), 7.63 (s, 1H), 6.89 (br s, 1H), 6.78 (br s, 1H), 6.36 (s, 2H), 3.29-3.20 (m, 2H), 1.14 (t, J=7.2 Hz, 3H). ¹³C NMR (125 MHz, d-DMSO) δ 166.30, 150.06, 143.50, 126.11, 111.26, 34.91, 14.43. HRMS (ESI) calc. for C₇H₁₂N₅O₂, [M+H]⁺: 198.0991, found: 198.0990.

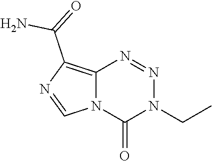

3-ethyl-4-oxo-3,4-dihydroimidazo[5,1-d][1,2,3,5]tetrazine-8-carboxamide (2). ¹H NMR (500 MHz, d-DMSO) δ 8.81 (s, 1H), 7.80 (br s, 1H), 7.67 (br s, 1H), 4.33 (q, J=7.2 Hz, 2H), 1.39 (t, J=7.2 Hz, 3H). ¹³C NMR (125 MHz, d-DMSO) δ 161.56, 138.78, 134.53, 130.45, 128.55, 44.23, 13.79. HRMS (ESI) calc. for C₇H₉N₆O₂, [M+H]⁺: 209.0787, found: 209.0784.

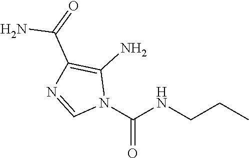

5-amino-N1-propyl-1H-imidazole-1,4-dicarboxamide (25). General procedure A. 59% yield as a pale gray solid. ¹H NMR (500 MHz, d-DMSO) δ 8.45 (t, J=5.5 Hz, 1H), 7.63 (s, 1H), 6.89 (br s, 1H), 6.77 (br s, 1H), 6.34 (s, 2H), 3.20-3.11 (m, 2H), 1.53 (sext, J=7.2 Hz, 2H), 0.88 (t, J=7.4 Hz, 3H). ¹³C NMR (125 MHz, d-DMSO) δ 166.33, 150.24, 143.53, 126.16, 111.28, 41.68, 22.10, 11.31. HRMS (ESI) calc. for C₈H₁₃N₅O₂Na, [M+Na]⁺: 234.0967, found: 234.0965.

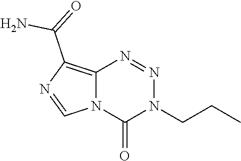 [SMILES: CCCn1nnc2c(C(N)=O)ncn2c1=O]

4-oxo-3-propyl-3,4-dihydroimidazo[5,1-d][1,2,3,5]tetrazine-8-carboxamide (3). General procedure A. 47% yield as a white solid. ¹H NMR (500 MHz, d-DMSO) δ 8.79 (s, 1H), 7.78 (br s, 1H), 7.66 (br s, 1H), 4.23 (t, J=7.1 Hz, 2H), 1.81 (sext, J=7.3 Hz, 2H), 0.94 (t, J=7.4 Hz, 3H). ¹³C NMR (125 MHz, d-DMSO) δ 161.57, 139.01, 134.47, 130.50, 128.61, 50.41, 21.61, 10.91. HRMS (ESI) calc. for CH₁N₆O₂, [M+H]⁺: 223.0943, found: 223.0952.

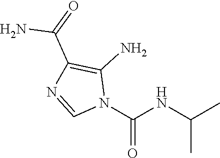

5-amino-N1-isopropyl-1H-imidazole-1,4-dicarboxamide (26). General procedure A. 69% yield as a white solid. ¹H NMR (500 MHz, d-DMSO) δ 8.23 (d, J=7.4 Hz, 1H), 7.65 (s, 1H), 6.89 (br s, 1H), 6.76 (br s, 1H), 6.33 (br s, 2H), 3.92 (sept, J=6.8 Hz, 1H), 1.17 (d, J=6.6 Hz, 6H). ¹³C NMR (125 MHz, d-DMSO) δ 166.32, 149.40, 143.53, 126.30, 111.27, 42.54, 22.05. HRMS (ESI) calc. for C₈H₁₃N₅O₂Na, [M+Na]⁺: 234.0967, 234.0966.

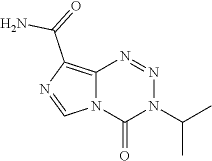 [SMILES: CC(C)n1nnc2c(C(N)=O)ncn2c1=O]

3-isopropyl-4-oxo-3,4-dihydroimidazo[5,1-d][1,2,3,5]tetrazine-8-carboxamide (4). General procedure A. 28% yield as a white solid. ¹H NMR (500 MHz, d-DMSO) δ 8.78 (s, 1H), 7.77 (br s, 1H), 7.66 (br s, 1H), 5.05 (sept, J=6.7 Hz, 1H), 1.49 (d, J=6.7 Hz, 6H). ¹³C NMR (125 MHz, d-DMSO) δ 161.56, 138.58, 134.21, 130.14, 128.57, 50.15, 21.13. HRMS (ESI) calc. for C₈H¹⁰N₆O₂Na, [M+Na]⁺: 245.0763, found: 245.0762.

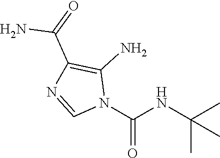 [SMILES: CC(C)(C)NC(=O)n1cnc(C(N)=O)c1N]

5-amino-N1-(tert-butyl)-1H-imidazole-1, 4-dicarboxamide (27)..General procedure A. 89% yield as a light purple solid. ¹H NMR (500 MHz, d-DMSO) δ 7.89 (s, 1H), 7.65 (s, 1H), 6.92 (br s, 1H), 6.78 (br s, 1H), 6.26 (br s, 2H), 1.37 (s, 9H). ¹³C NMR (125 MHz, d-DMSO) δ 166.39, 149.11, 143.35, 127.20, 111.47, 51.70, 28.26. FIRMS (ESI) calc. for C₉H₁₆N₅O₂, [M+H]⁺: 226.1304, 226.1313.

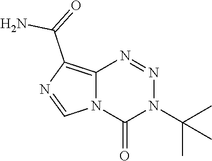 [SMILES: CC(C)(C)n1nnc2c(C(N)=O)ncn2c1=O]

3-(tert-butyl)-4-oxo-3,4-dihydroimidazo[5,1-d][1,2,3,5]tetrazine-8-carboxamide (5). General procedure A. 4% yield as a white solid. ¹H NMR (500 MHz, d-DMSO) δ 8.75 (s, 1H), 7.75 (br s, 1H), 7.63 (br s, 1H), 1.70 (s, 9H). ¹³C NMR (500 MHz, d-DMSO) δ 161.70, 138.49, 134.31, 129.63, 128.13, 64.35, 28.27. HRMS (ESI) calc. for C₉H₁₂N₆O₂Na, [M+Na]⁺: 259.0919, found: 259.0918.

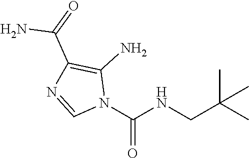 [SMILES: CC(C)(C)CNC(=O)n1cnc(C(N)=O)c1N]

5-amino-N1-neopentyl-1H-imidazole-1,4-dicarboxamide (28). General procedure A. 49% yield as a white solid. ¹H NMR (500 MHz, d-DMSO) δ 8.39 (t, J=6.2 Hz, 1H), 7.71 (s, 1H), 6.92 (br s, 1H), 6.79 (br s, 1H), 6.32 (br s, 2H), 3.04 (d, J=6.2 Hz, 2H), 0.88 (s, 9H). ¹³C NMR (125 MHz, d-DMSO) δ 166.36, 150.73, 143.52, 126.49, 111.41, 50.88, 32.55, 27.29. HRMS (ESI) calc. for C₁₀H₁₈N₅O₂, [M+H]⁺: 240.1460, found: 240.1455.

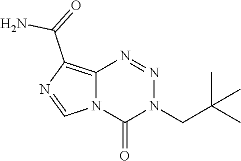 [SMILES: CC(C)(C)Cn1nnc2c(C(N)=O)ncn2c1=O]

3-neopentyl-4-oxo-3,4-dihydroimidazo[5,1-d][1,2,3,5]tetrazine-8-carboxamide (6). General procedure A. 17% yield as a white solid. H NMR (500 MHz, d-DMSO) δ 8.78 (s, 1H), 7.80 (br s, 1H), 7.66 (br s, 1H), 4.10 (s, 2H), 0.98 (s, 9H). ¹³C NMR (500 MHz, d-DMSO) δ 161.58, 139.72, 134.32, 130.59, 128.80, 58.57, 33.57, 27.34. HRMS (ESI) calc. for C₁₀H₁₅N₆O₂, [M+H]⁺: 251.1256, found: 251.1256.

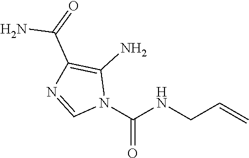

N1-allyl-5-amino-1H-imidazole-1,4-dicarboxamide (29). General procedure A. 63% yield as a beige solid. ¹H NMR (500 MHz, d-DMSO) δ 8.68 (t, J=5.7 Hz, 1H), 7.66 (s, 1H), 6.88 (br s, 1H), 6.77 (br s, 1H), 6.35 (br s, 2H), 5.94-5.74 (m, 1H), 5.26-5.18 (m, 1H), 5.16-5.09 (m, 1H), 3.85 (t, J=5.21 Hz, 2H). ¹³C NMR (125 MHz, d-DMSO) δ 166.34, 150.18, 143.58, 134.31, 126.17, 116.04, 111.26, 42.14. HRMS (ESI) calc. for C₈H₁₂N₅O₂, [M+H]⁺: 210.0991, found: 210.0986.

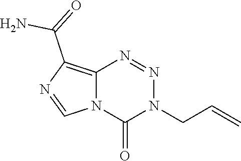 [SMILES: C=CCn1nnc2c(C(N)=O)ncn2c1=O]

3-allyl-4-oxo-3,4-dihydroimidazo[5,1-d][1,2,3,5]tetrazine-8-carboxamide (9). General procedure A. 23% yield as a pale yellow solid. ¹H NMR (500 MHz, d-DMSO) δ 8.83 (s, 1H), 7.81 (br s, 1H), 7.69 (br s, 1H), 6.09-5.97 (m, 1H), 5.39-5.31 (m, 1H), 5.29-5.22 (m, 1H), 4.92 (d, J=5.4 Hz, 2H). ¹³C NMR (125 MHz, d-DMSO) δ 161.51, 138.87, 134.45, 131.91, 130.77, 128.81, 118.17, 50.80. HRMS (ESI) calc. for C₈H₈N₆O₂Na, [M+Na]⁺: 243.0606, found: 243.0603.

### General Procedure B.

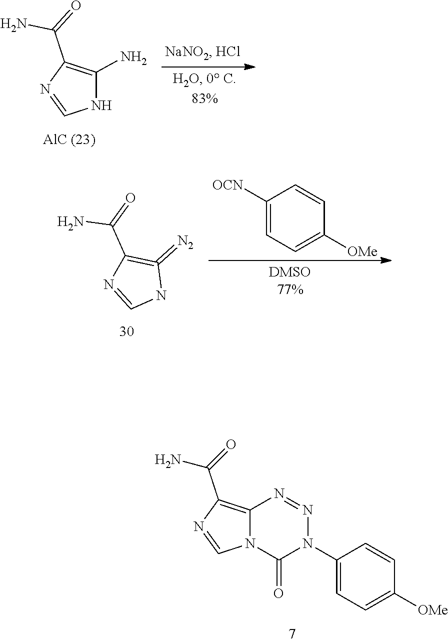

In a 300 mL round bottom flask, NaNO₂ (1.40 g, 20.3 mmol, 1.1 eq.) was dissolved in water (28 mL, 0.66 M). In a separate flask, 4-aminoimidazole-5-carboxamide (23, 3.01 g, 18.5 mmol, 1 eq.) was completely dissolved in 1 μM HCl (28 mL, 0.66 M) until translucent. This solution was then added slowly to the aqueous solution of NaNO₂ in the dark at 0° C. and stirred vigorously for 10 min. The precipitate was filtered, washed with water and then diethyl ether to afford 4-diazoimidazole-5-carboxamide (30) as a beige solid (2.106 g, 15.4 mmol, 83% yield).

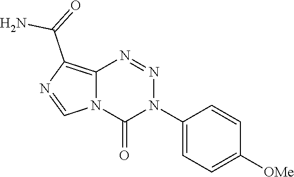 [SMILES: COc1ccc(-n2nnc3c(C(N)=O)ncn3c2=O)cc1]

3-(4-methoxyphenyl)-4-oxo-3,4-dihydroimidazo[5,1-d][1,2, 3,S]tetrazine-8-carboxamide (7). To an oven dried 25 mL round bottom flask, diazo 30 (205.7 mg, 1.50 mmol, 1 eq.) and dry DMSO (3.3 mL, 0.45 M) were added. Next, 4-methoxyphenyl isocyanate (0.23 mL, 1.8 mmol, 1.2 eq.) was added in the dark. The reaction was stirred overnight protected from light. Upon completion, the reaction mixture was poured into ˜10 mL ice and water. The resultant solid was filtered, washing with water and diethyl ether to afford a pale orange solid. The crude solid (containing a mixture of N, N′-bis-(4-methoxyphenyl)urea and the desired product) was dissolved completely in DMF until translucent. Water was added dropwise to precipitate a white solid, which was filtered and washed with water and diethyl ether to afford 332.4 mg of 7 (1.16 mmol, 77% yield). ¹H NMR (500 MHz, d-DMSO) δ 8.92 (s, 1H), 7.87 (s, 1H), 7.73 (s, 1H), 7.54 (d, J=8.9 Hz, 2H), 7.15 (d, J=8.9 Hz, 2H), 3.84 (s, 3H). ¹³C NMR (125 MHz, d-DMSO) δ 161.45, 159.77, 139.06, 134.09, 131.09, 129.86, 129.33, 128.18, 114.32, 55.55. HRMS (ESI) calc. for C₁₂H₁₀N₆O₃Na, [M+Na]⁺: 309.0712, found: 309.0711.

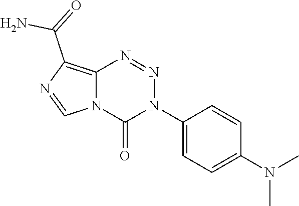 [SMILES: CN(C)c1ccc(-n2nnc3c(C(N)=O)ncn3c2=O)cc1]

3-(4-(dimethylamino)phenyl)-4-oxo-3,4-dihydroimidazo[5,1-d][1,2,3,5]tetrazine-8-carboxamide (8). General procedure B. 65% yield as a light brown solid. ¹H NMR (400 MHz, d-DMSO) δ 8.98 (s, 1H), 7.86 (br s, 1H), 7.71 (br s, 1H), 7.40 (d, J=9.0 Hz, 2H), 6.85 (d, J=9.1 Hz, 2H), 2.99 (s, 6H). ¹³C NMR (500 MHz, d-DMSO) δ 161.53, 150.72, 139.11, 134.16, 130.83, 129.14, 127.42, 125.53, 111.79, 40.77. HRMS (ESI) calc. for C₁₃H₁₃N₇O₂Na, [M+Na]⁺: 322.1028, found: 322.1021.

### General Procedure C.

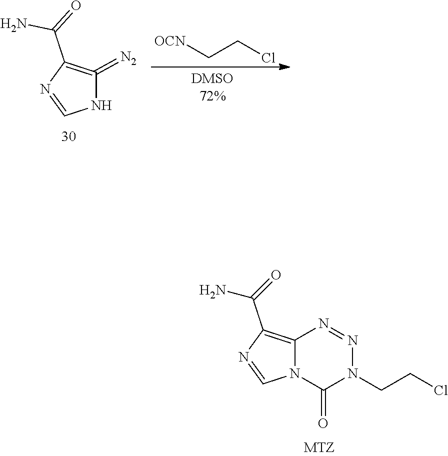

To an oven-dried 25 mL round bottom flask, was added diazo 30 (142.0 mg, 1.0 mmol, 1 eq.) followed by anhydrous DMSO (2.3 mL, 0.45 M). Next, 2-chloroethyl isocyanate (0.11 mL, 1.29 mmol, 1.2 eq.) was added in the dark and the reaction was stirred overnight under nitrogen. Upon completion, the reaction mixture was poured into ice and water and stirred for five minutes. The resultant solid was filtered, washing with water and diethyl ether to afford MTZ (180.8 mg, 0.75 mmol, 72% yield) as a pale orange solid.

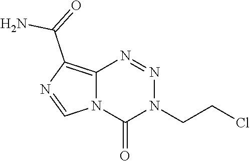 [SMILES: NC(=O)c1ncn2c(=O)n(CCCl)nnc12]

3-(2-chloroethyl)-4-oxo-3,4-dihydroimidazo[5,1-d][1,2,3,5]tetrazine-8-carboxamide (MTZ). ¹H NMR (500 MHz, d-DMSO) δ 8.87 (s, 1H), 7.84 (br s, 1H), 7.70 (br s, 1H), 4.64 (t, J=6.1 Hz, 2H), 4.03 (t, J=6.1 Hz, 2H). ¹³C NMR (125 MHz, d-DMSO) δ 161.37, 139.07, 134.03, 131.15, 129.08, 49.95, 41.46.

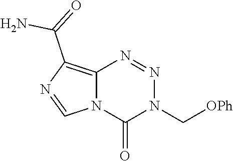 [SMILES: NC(=O)c1ncn2c(=O)n(COc3ccccc3)nnc12]

4-oxo-3-(phenoxymethyl)-3,4-dihydroimidazo[5,1-d][1,2,3,5]tetrazine-8-carboxamide (11). General procedure C. 79% yield as a pale pink solid. ¹H NMR (500 MHz, d-DMSO) δ 8.90 (s, 1H), 7.86 (br s, 1H), 7.73 (br s, 1H), 7.38-7.31 (m, 2H), 7.21-7.14 (m, 2H), 7.06 (tt, J=7.4, 1.0 Hz, 1H), 6.26 (s, 2H). ¹³C NMR (125 MHz, d-DMSO) δ 161.29, 156.25, 139.15, 133.84, 131.80, 129.86, 129.66, 122.54, 116.46, 75.66. HRMS (ESI) calc. for C₁₂H₁₁N₆O₃, [M+H]⁺: 287.0893, found: 287.0890.

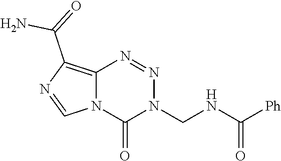 [SMILES: NC(=O)c1ncn2c(=O)n(CNC(=O)c3ccccc3)nnc12]

3-(benzamidomethyl)-4-oxo-3,4-dihydroimidazo[5,1-d][1,2,3,5]tetrazine-8-carboxamide (12). General procedure C. 8% yield as a white solid. ¹H NMR (500 MHz, d-DMSO) δ 9.47 (t, J=5.9 Hz, 1H), 8.87 (s, 1H), 7.91-7.86 (m, 2H), 7.82 (br s, 1H), 7.70 (br s, 1H), 7.56 (tt, J=7.4, 1.3 Hz, 1H), 7.50-7.44 (m, 2H), 5.82 (d, J=5.9 Hz, 2H). ¹³C NMR (125 MHz, d-DMSO) δ 166.55, 161.37, 138.66, 134.05, 133.26, 131.83, 131.07, 129.17, 128.35, 127.53, 53.45. HRMS (ESI) calc. for C₁₃H₁₂N₇O₃, [M+H]⁺: 314.1002, found: 314.0996.

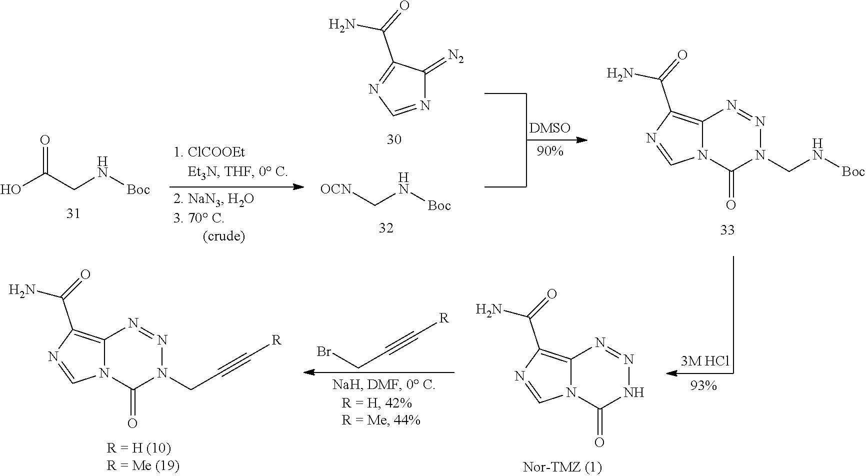

tert-butyl (nitrosomethyl)carbamate (32). In a 200 mL round bottom flask, Boc-Gly-OH (31, 3 g, 17.1 mmol, 1 eq.) was dissolved in THF (62 mL, 0.28 M). The mixture was cooled to 0° C. before ethyl chloroformate (1.75 mL, 18.3 mmol, 1.07 eq.) and triethylamine (2.6 mL, 18.6 mmol, 1.1 eq.) were added and stirred at 0° C. After 1 hour, a solution of sodium azide (1.67 g, 25.7 mmol, 1.1 eq.) in water (15 mL, 1.1 M) was added and the mixture was stirred an additional hour at 0° C. Upon completion, the reaction was stopped and transferred to a separatory funnel. The organic layer was collected, and the aqueous layer was extracted with toluene (×4). The combined organic layers were washed with saturated aqueous sodium bicarbonate (×2), 1 μM HCl (xl), and water (xl) before being dried over magnesium sulfate. The crude azide solution was heated slowly to 55° C. in an oil bath with gentle stirring. When nitrogen evolution was no longer observable, the reaction was heated further to 70° C. and stirred for another 2 hours. The reaction was then cooled to ambient temperature and concentrated to −2 mL to afford a solution of 32 in toluene. Yields of ˜90% are consistently obtained as gauged by ¹H NMR.

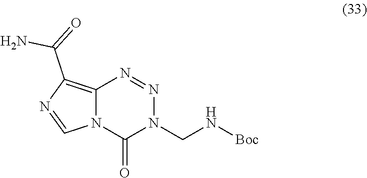 [SMILES: CC(C)(C)OC(=O)NCn1nnc2c(C(N)=O)ncn2c1=O]

Tert-butyl ((8-carbamoyl-4-oxoimidazo[5,1-d][1,2,3,5]tetrazin-3(4H)-yl)methyl)carbamate (33). To an oven dried 65 mL round bottom flask, diazo 30 (1.52 g, 11.1 mmol, 1 eq.) and dry DMSO (23 mL, 0.48 M) were added. Next, crude isocyanate 32 was added (as a solution in toluene) in the dark. The reaction was stirred overnight protected from light. Upon completion, the mixture was poured into ˜200 mL ice and water. The resultant solid was filtered, washed with water and then diethyl ether to afford 33 as a pink solid (3.0921 g, 10.0 mmol, 90% yield). ¹H NMR (500 MHz, d-DMSO) δ 8.84 (s, 1H), 8.04 (br s, 1H), 7.82 (br s, 1H), 7.70 (br s, 1H), 5.49 (d, J=6.4 Hz, 2H), 1.38 (s, 9H). ¹³C NMR (500 MHz, d-DMSO) δ 161.50, 155.03, 138.65, 134.16, 130.99, 129.16, 78.98, 54.91, 28.08. HRMS (ESI) calc. for C₁₁H₁₆N₇O₄, [M+H]⁺: 310.1264, found: 310.1254.

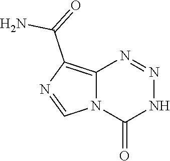 [SMILES: NC(=O)c1ncn2c(=O)[nH]nnc12]

4-oxo-3,4-dihydroimidazo[5,1-d][1,2,3,5]tetrazine-8-carboxamide (Nor-TMZ, 1). In a 300 mL round bottom flask, 33 (3.195 g, 10.3 mmol, 1 eq.) was suspended in 3 μM HCl (150 mL, 0.07 M) and stirred overnight at room temperature. Upon completion, the reaction was stopped and the round bottom was submerged in an ice bath for 1 hour. The resultant solid was filtered, washing with water and diethyl ether to afford Nor-TMZ (1) as alight pink solid (1.7277 g, 9.6 mmol, 93%). ¹H NMR (500 MHz, d-DMSO) δ 14.97 (br s, 1H), 8.77 (s, 1H), 7.77 (br s, 1H), 7.66 (br s, 1H). ¹³C NMR (125 MHz, d-DMSO) δ 161.62, 139.07, 134.51, 130.37, 128.53. HRMS (ESI) calc. for C₅H5N₆02, [M+H]⁺: 181.0474, found: 181.0467.

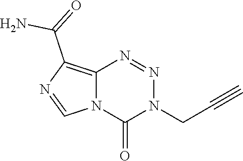 [SMILES: C#CCn1nnc2c(C(N)=O)ncn2c1=O]

4-oxo-3-(prop-2-yn-1-yl)-3,4-dihydroimidazo[5,1-d][1,2,3,5]tetrazine-8-carboxamide (10). To an oven dried 100 mL round bottom flask, Nor-TMZ (1.17 g, 6.5 mmol, 1 eq.) was added followed by anhydrous DMF (21.5 mL, 0.3 M). The reaction was cooled to 0° C. and sodium hydride (60% w/w in mineral oil, 297.4 mg, 7.4 mmol, 1.1 eq.) was added under nitrogen. The reaction was stirred at 0° C. for 20 min before propargyl bromide (80% w/w in toluene, 2.5 mL, 23.2 mmol, 3.6 eq.) was added. The reaction was stirred 0° C. to room temperature overnight. Upon completion, the reaction mixture was concentrated and purified by flash silica chromatography (100% ethyl acetate) to afford 10 as a light brown solid (597.0 mg, 2.74 mmol, 42% yield). ¹H NMR (500 MHz, d-DMSO) δ 8.87 (s, 1H), 7.85 (br s, 1H), 7.72 (br s, 1H), 5.14 (d, J=2.5 Hz, 2H), 3.53 (t, J=2.4 Hz, 1H). ¹³C NMR (125 MHz, d-DMSO) δ 161.38, 138.55, 134.20, 131.20, 129.20, 77.34, 76.49, 38.47. HRMS (ESI) calc. for C₈H₇N₆O₂, [M+H]⁺: 219.0630, found: 219.0622.

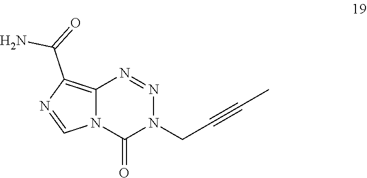 [SMILES: CC#CCn1nnc2c(C(N)=O)ncn2c1=O]

3-(but-2-yn-1-yl)-4-oxo-3,4-dihydroimidazo[5,1-d][1,2,3,5]tetrazine-8-carboxamide (19). To an oven dried 25 mL round bottom flask, Nor-TMZ (200.6 mg, 1.11 mmol, 1 eq.) was added followed by anhydrous DMF (7.5 mL, 0.15 M). The reaction was cooled to 0° C. and sodium hydride (60% w/w in mineral oil, 53.8 mg, 1.34 mmol, 1.2 eq.) was added under nitrogen. The reaction was stirred at 0° C. for 20 min before 1-bromo-2-butyne (0.34 mL, 3.9 mmol, 3.5 eq.) was added. The reaction was stirred 0° C. to room temperature overnight. Upon completion, the reaction mixture was concentrated and purified by flash silica chromatography (100% ethyl acetate) to afford 19 as a light brown solid (113.2 mg, 0.49 mmol, 44% yield). ¹H NMR (500 MHz, d-DMSO) δ 8.84 (s, 1H), 7.83 (br s, 1H), 7.71 (br s, 1H), 5.10-5.05 (q, J=2.4 Hz, 2H), 1.83 (t, J=2.5 Hz, 3H). ¹³C NMR (125 MHz, d-DMSO) δ 161.37, 138.49, 134.20, 131.19, 129.08, 81.65, 72.87, 38.88, 3.13.

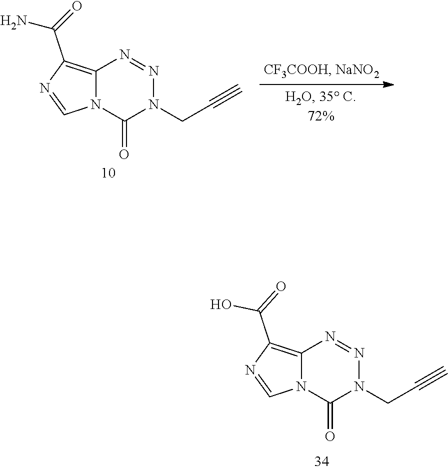 [SMILES: C#CCn1nnc2c(C(N)=O)ncn2c1=O ; C#CCn1nnc2c(C(=O)O)ncn2c1=O]

4-oxo-3-(prop-2-yn-1-yl)-3,4-dihydroimidazo[5,1-d][1,2,3,5]tetrazine-8-carboxylic acid (34). To a 20 mL vial, 10 (482 mg, 2.2 mmol, 1 eq.) was added and suspended in trifluoroacetic acid (2.2 mL, 1M). Sodium nitrite (528.7 mg, 7.66 mmol, 3.5 eq.) was dissolved in water (1.1 mL, 2 M) and added slowly over 20 min to the acidic solution of 10. When addition was complete, the reaction was heated to 35° C. After 3 hours, the reaction was stopped and cooled to room temperature. While stirring, ice was added to the crude suspension to precipitate an orange solid, which was filtered and washed with ice-cold water and diethyl ether to afford pure 34 (346.0 mg, 1.58 mmol, 72% yield). ¹H NMR (500 MHz, d-DMSO) δ 8.83 (s, 1H), 5.13 (d, J=2.5 Hz, 2H), 3.53 (t, J=2.5 Hz, 1H). ¹³C NMR δ 161.78, 138.45, 135.92, 129.77, 129.04, 77.26, 76.58, 38.60. HRMS (ESI) calc. for C₈H₅N₅O₃, M⁺: 219.0392, found: 219.0624.

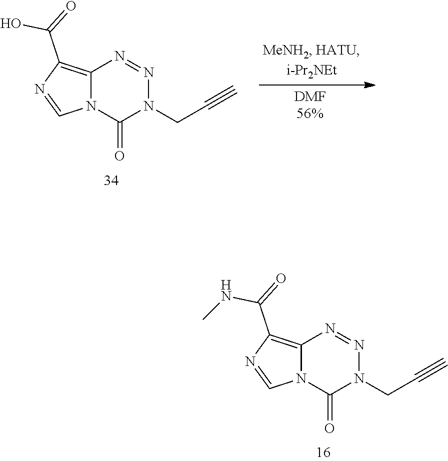 [SMILES: C#CCn1nnc2c(C(=O)O)ncn2c1=O ; C#CCn1nnc2c(C(=O)NC)ncn2c1=O]

N-methyl-4-oxo-3-(prop-2-yn-1-yl)-3,4-dihydroimidazo[5,1-d][1,2,3,5]tetrazine-8-carboxamide (16). To an oven dried vial, 34 (30.3 mg, 0.138 mmol, 1 eq.), methylamine hydrochloride (10.7 mL, 0.16 mmol, 1.1 eq.), and HATU (64.2 mg, 0.169 mmol, 1.2 eq.) were added and suspended in dry DMF (0.7 mL, 0.2 M). Next, under nitrogen, diisopropylethylamine (0.05 mL, 0.29 mmol, 2.1 eq.) was added slowly and the reaction was stirred overnight at room temperature. Upon completion, the reaction was concentrated and the crude mixture was purified by flash silica chromatography (1:1 hexanes:ethyl acetate) to afford 16 as a white solid (17.9 mg, 0.077 mmol, 56% yield). ¹H NMR (500 MHz, CDCl₃) δ 8.35 (s, 1H), 7.25 (br s, 1H), 5.10 (d, J=2.6 Hz, 2H), 3.01 (d, J=5.0 Hz, 3H), 2.45 (t, J=2.5 Hz, 1H). ¹³C NMR (125 MHz, CDCl₃) δ 159.65, 138.00, 133.21, 132.59, 128.22, 75.46, 74.52, 39.04, 26.04. HRMS (ESI) calc. for C₉H₉N₆O₂, [M+H]⁺: 233.0787, found: 233.0793.

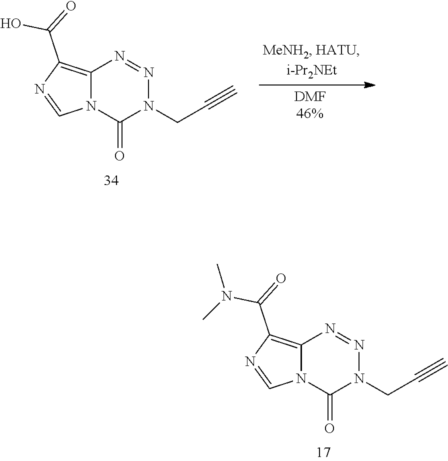 [SMILES: C#CCn1nnc2c(C(=O)O)ncn2c1=O ; C#CCn1nnc2c(C(=O)N(C)C)ncn2c1=O]

N,N-dimethyl-4-oxo-3-(prop-2-yn-1-yl)-3,4-dihydroimidazo[5,1-d][1,2,3,5]tetrazine-8-carboxamide (17). To an oven dried vial, 34 (30.3 mg, 0.138 mmol, 1 eq.), dimethylamine (2 M solution in THF, 0.075 mL, 0.15 mmol, 1.1 eq.), and HATU (62.4 mg, 0.169 mmol, 1.2 eq.) were added and suspended in dry DMF (0.7 mL, 0.2 M). Next, under nitrogen, diisopropylethylamine (0.05 mL, 0.29 mmol, 2.1 eq.) was added slowly and the reaction was stirred overnight at room temperature. Upon completion, the reaction was concentrated and the crude mixture was purified by flash silica chromatography (5:2 ethyl acetate:hexanes) to afford 17 as a white solid (15.5 mg, 0.063 mmol, 46% yield). ¹H NMR (500 MHz, CDCl₃) δ 8.44 (s, 1H), 5.14 (d, J=2.5 Hz, 2H), 3.22 (s, 3H), 3.19 (s, 3H), 2.44 (t, J=2.2 Hz, 1H). ¹³C NMR (125 MHz, CDCl₃) δ 161.51, 138.21, 134.92, 133.50, 128.23, 75.72, 74.46, 38.96, 38.65, 35.78. HRMS (ESI) calc. for C₁₀H₁₁N₆O₂, [M+H]⁺: 247.0943, found: 247.0938.

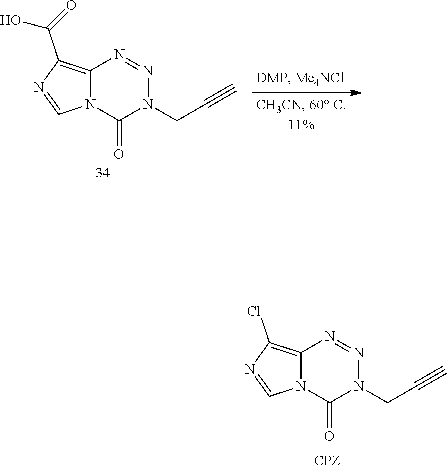 [SMILES: C#CCn1nnc2c(C(=O)O)ncn2c1=O ; C#CCn1nnc2c(Cl)ncn2c1=O]

8-chloro-3-(prop-2-yn-1-yl)imidazo[5,1-d][1,2,3,5]tetrazin-4(3H)-one (18, CPZ). To an oven dried vial, 34 (79.7 mg, 0.36 mmol, 1 eq.), Dess-Martin periodinane (611.2 mg, 1.44 mmol, 4 eq.) and tetramethylammonium chloride (102.3 mg, 0.94 mmol, 2.6 eq.) were added and suspended in dry acetonitrile (1.8 mL, 0.2 M) under nitrogen. The reaction was heated at 60° C. for 4 hours before the solvent was evaporated and the crude solid was purified by flash silica chromatography (4:1 hexanes:ethyl acetate) to afford CPZ as a white solid (8.3 mg, 0.04 mmol, 11% yield). ¹H NMR (500 MHz, CDCl₃) δ 8.37 (s, 1H), 5.11 (d, J=2.5 Hz, 2H), 2.44 (t, J=2.5 Hz, 1H). ¹³C NMR (125 MHz, CDCl₃) δ 138.06, 130.68, 130.56, 127.96, 75.88, 74.59, 38.98. HRMS (EI) calc. for C₇H₄N₅OCl, M⁺: 209.0104, found: 209.0110.

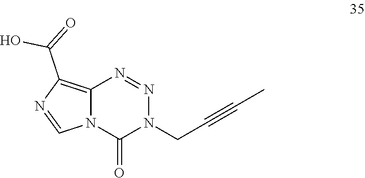 [SMILES: CC#CCn1nnc2c(C(=O)O)ncn2c1=O]

3-(but-2-yn-1-yl)-4-oxo-3,4-dihydroimidazo[5,1-d][1,2,3,5]tetrazine-8-carboxylic acid. To a 20 mL vial, 19 (482 mg, 2.08 mmol, 1 eq.) was added and suspended in trifluoroacetic acid (2.1 mL, 1M). Sodium nitrite (501.3 mg, 7.265 mmol, 3.5 eq.) was dissolved in water (1.1 mL, 2 M) and added slowly over 20 min to the acidic solution of 19. When addition was complete, the reaction was heated to 35° C. overnight. The reaction was stopped and cooled to room temperature. While stirring, ice was added to the crude suspension to precipitate an orange solid, which was filtered and washed with ice-cold water and diethyl ether to afford pure 35 (319 mg, 1.37 mmol, 66% yield).

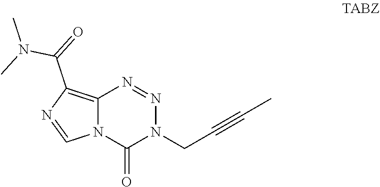 [SMILES: CC#CCn1nnc2c(C(=O)N(C)C)ncn2c1=O]

3-(but-2-yn-1-yl)-N,N-dimethyl-4-oxo-3,4-dihydroimidazo[5,1-d][1,2,3,5]tetrazine-8-carboxamide (TABZ). To an oven dried vial, 35 (206 mg, 0.884 mmol, 1 eq.) was suspended in dry acetonitrile (3.53 mL, 0.25 M). DIPEA (0.31 mL, 1.768 mmol, 2 eq.) was added under nitrogen and the reaction solubilized and stirred for 10 min. T3P (50% in ethyl acetate, 0.62 mL, 1.061 mmol, 1.2 eq.) was added and the reaction stirred an additional 10 min. Finally, dimethylamine (2 μM solution in THF, 0.44 mL, 0.884 mmol, 1 eq.) was added and the reaction stirred overnight at room temperature. The reaction was subsequently diluted with ethyl acetate and washed with water. The organic layer was dried with MgSO4 and then concentrated under reduced pressure. Crude TABZ was purified by reverse phase chromatography (95% water, 5% acetonitrile) to obtain TABZ as an off-white solid (70 mg, 0.2700 mmol, 30% yield).

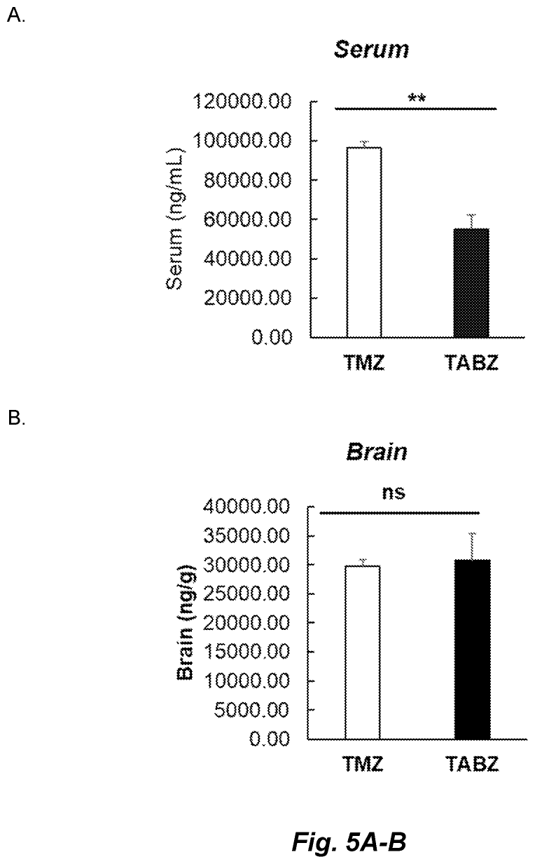

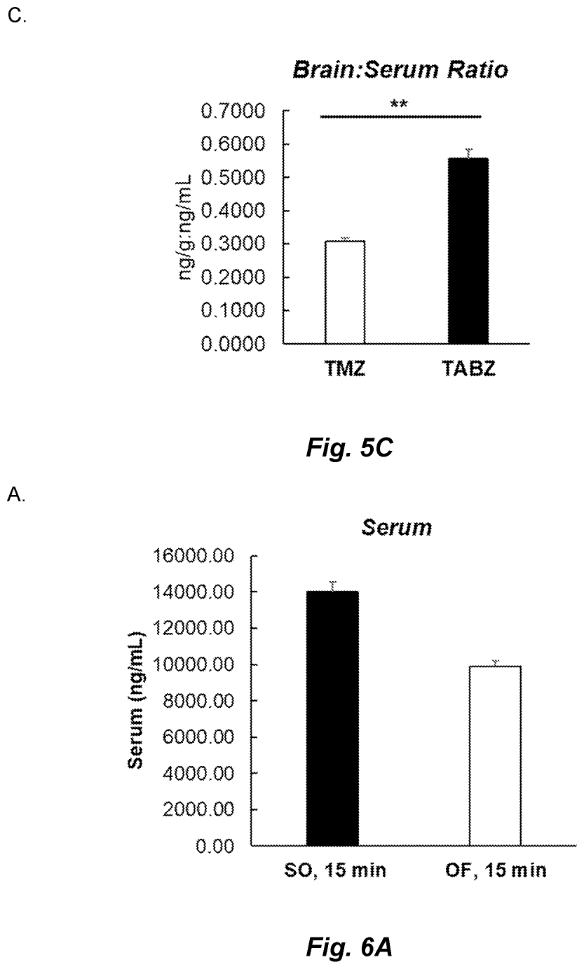

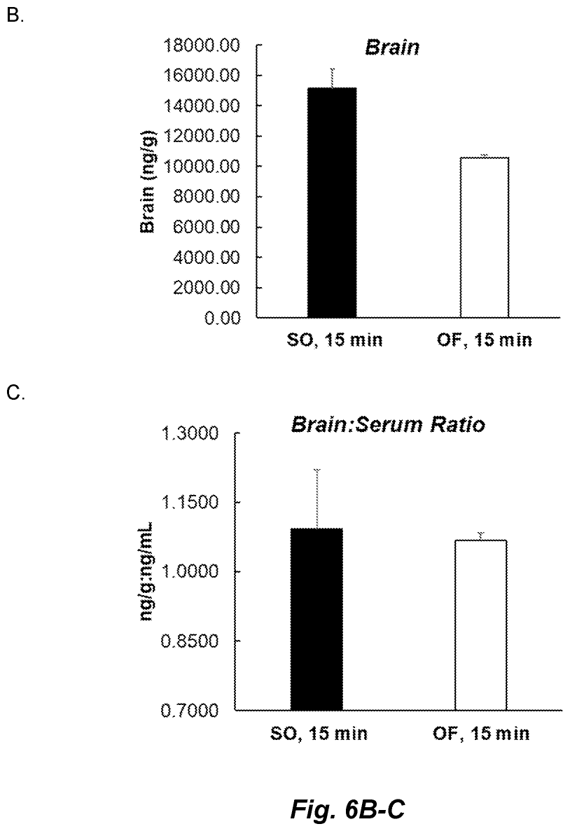

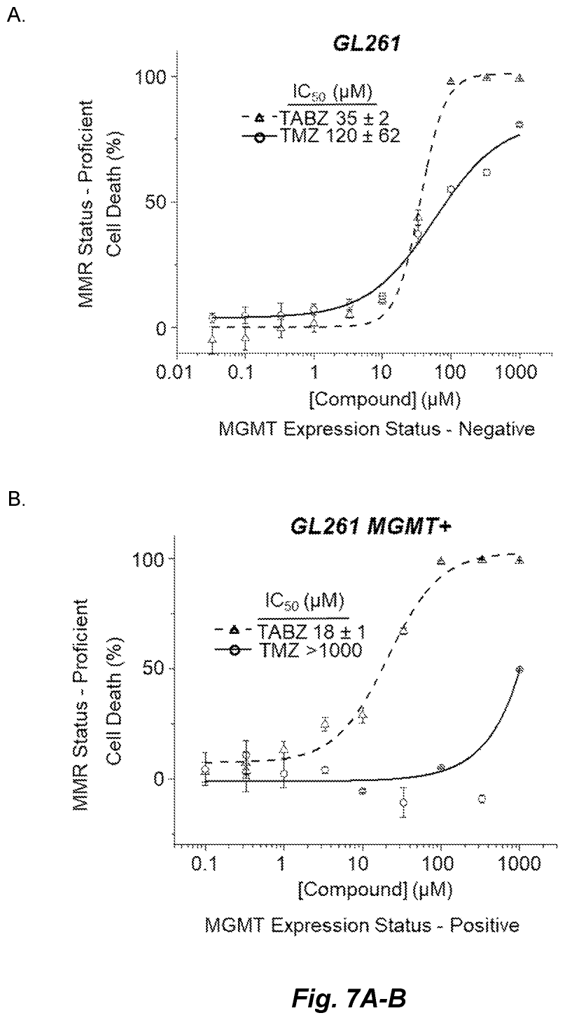

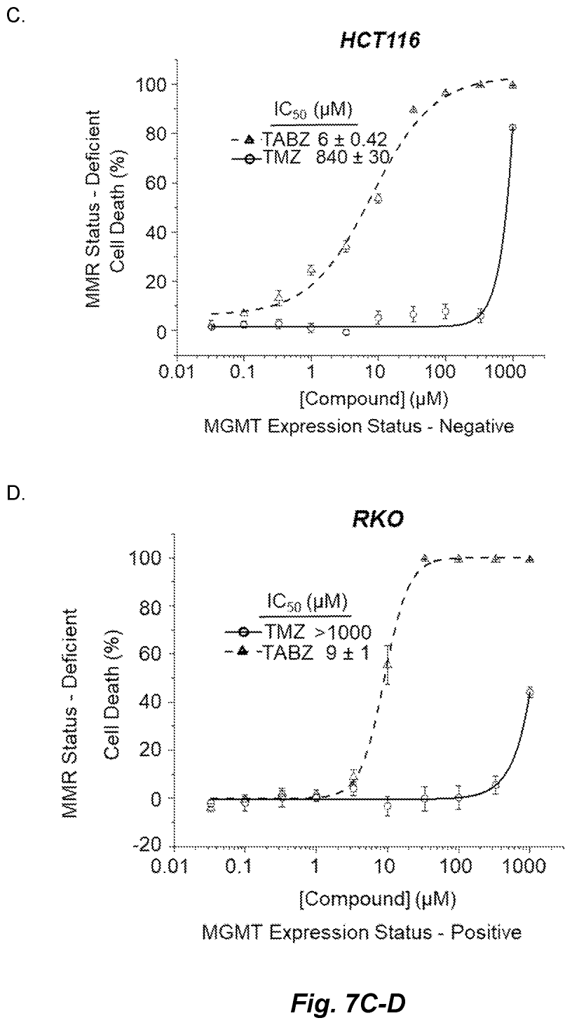

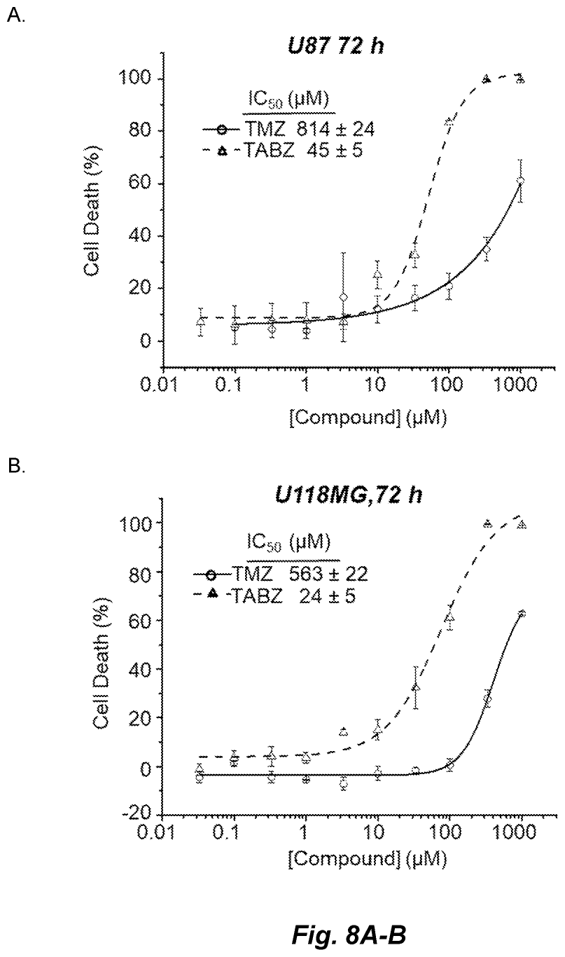

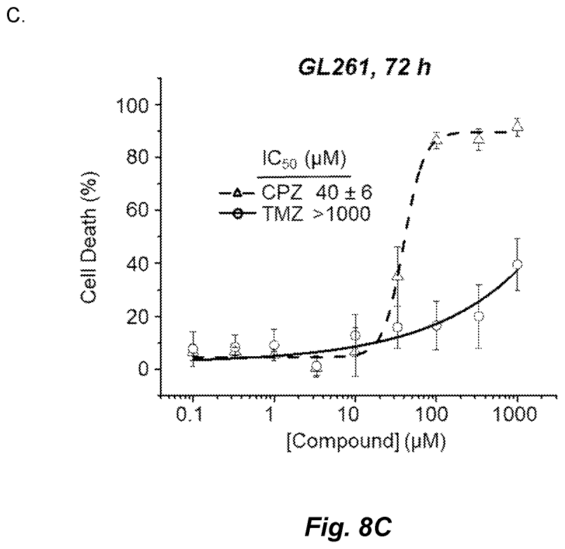

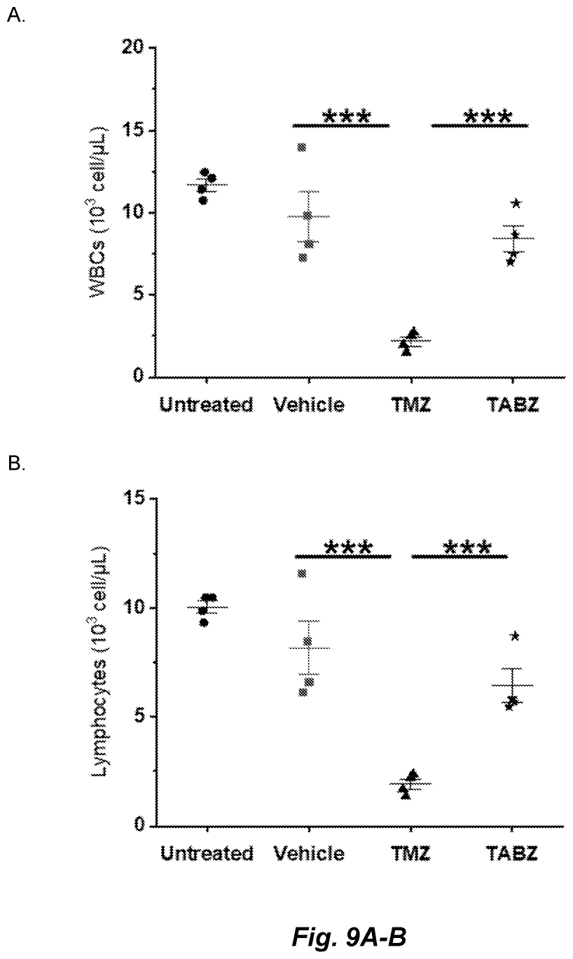

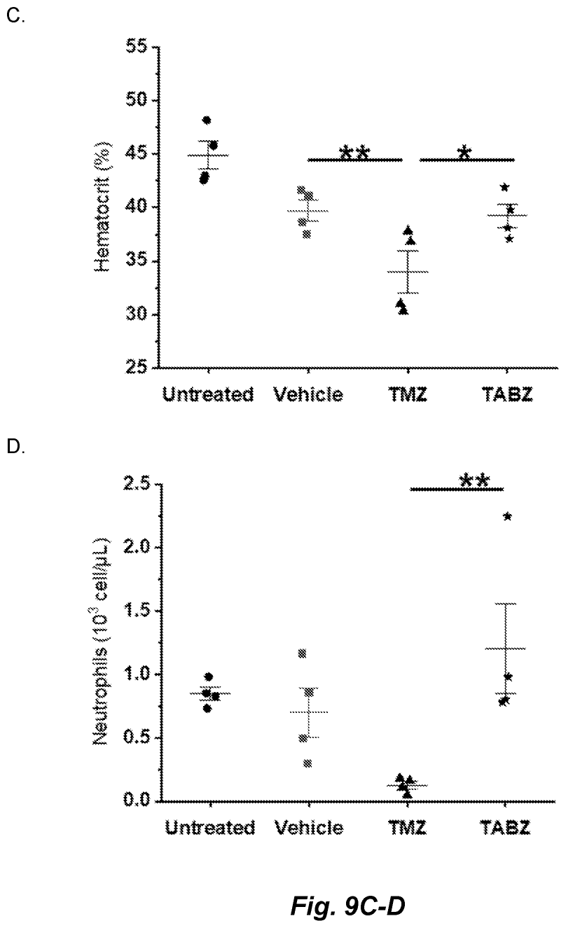

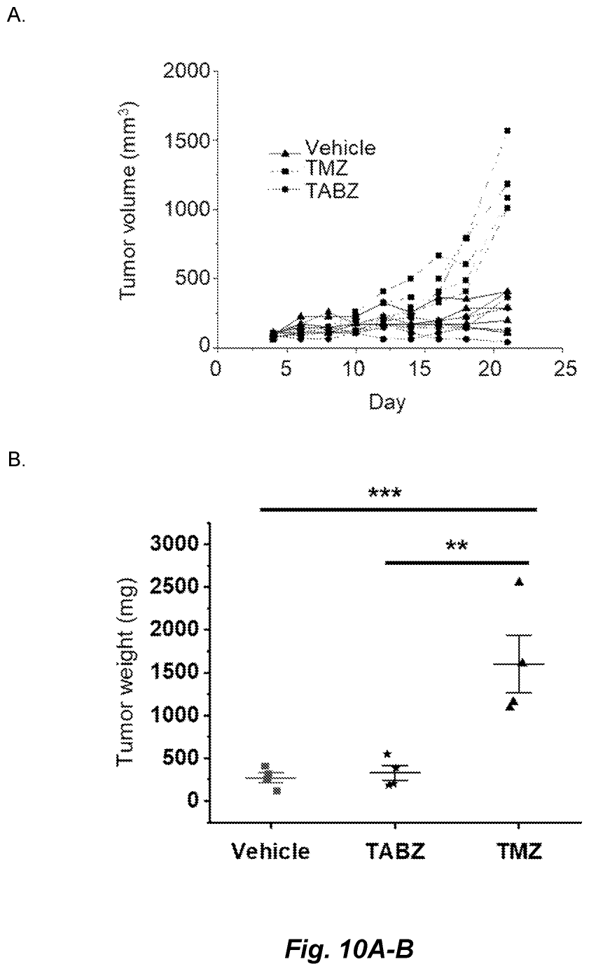

### Example 3. TABZ Comparative Data (FIG. 5 to FIG. 10)

TABLE 5
IV Formulations for TMZ, TABZ, and CPZ.

| Formulation | TMZ | TABZ | CPZ |
|---|---|---|---|
| 15/10/75 DMSO/NMP/ | Soluble | Soluble 20 min | Insoluble |
| 20% SPE-βCD | | | |
| 15/7/5/73 DMSO/NMP/ | Soluble | Soluble >25 min | Insoluble |
| T20/20% SPE-βCD | | | |
| 20/80 DMSO/PBS* | Soluble* | Soluble* | Insoluble |

Vehicle: 6.6 mg/mL solution for 200 μL injection.
*Vehicle: 5 mg/mL solution for 100 μL injection.

TABLE 6
Tolerability in mice. Dosing once per day over 5 days.

| | Administration | | | | | |
|---|---|---|---|---|---|---|
| | I.P. | | | I.V. | | |
| Compound | TMZ | CPZ | TABZ | TMZ | CPZ | TABZ |
| MTD (mg/kg) | 66 | 66 | 66 | 66 | N/A | 66 |
| | mg/kg | mg/kg | mg/kg | mg/kg | | mg/kg |

Formulations: For CPZ and TMZ assessed head-to-head in a mixture of 35/25/6/34 PEG400/propylene glycol/Tween80/sterile water (6.6 mg/mL solution).
For TABZ and TMZ assessed head-to-head in a mixture of 15/15/70 DMSO/propylene glycol/PBS (6.6 mg/mL solution). TABZ is able to be dosed 2×/day over 5 days at 66 mg/kg.

TABLE 7
Hepatic Metabolism of TABZ.

| | Stability | Stability |
|---|---|---|
| Compound | (2 h, with MLM) | (2 h, no MLM) |
| TMZ | 95 ± 1% | 92 ± 0.2% |
| propranolol | 82 ± 0.2% | 104 ± 2% |
| N-Me-TMZ | 86 ± 1% | 93 ± 1% |
| N,N-DiMe-TMZ | 81 ± 2% | 92 ± 3% |
| TABZ | 77 ± 2% | 87 ± 2% |
| But | 89 ± 3% | 85 ± 1% |

The metabolic stability was assessed in mouse liver microsomes. Compounds were incubated with microsomes for 2 h before the percentage remaining was quantified relative to t0 via LC-MS/MS. Experiments assessing stability in the absence of microsomes were identical but replaced liver microsomes with PBS. Error is SEM, n ≥ 2. Propranolol was used as the positive control for hepatic metabolism.

TABLE 8
Comparing Brain Tissue Binding between TABZ and TMZ.

| Homogenization Method | Fraction | TABZ | TMZ |
|---|---|---|---|
| Ultrasonication | Soluble | 61 ± 4% | 66 ± 3% |
| | Insoluble | 39 ± 4% | 30 ± 3% |
| Tisssue Homogenizer | Soluble | 64 ± 4% | 59 ± 5% |
| | Insoluble | 40 ± 4% | 40 ± 5% |

Incubated compound (final concentration 10 uM in 1% DMSO) in 200 uL of brain tissue homogenate. Incubated for 1 h at 37 C. shaking at 250 rpm. Centrifuged at max for 30 min and isolated 25 uL of supernatant which was acidified with 8.5% H3PO4. Added 1:1 ACN to remaining sample, then repeated isolation process. Submitted to LC-MS/MS analysis, looking for intact compound.

### Example 4. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 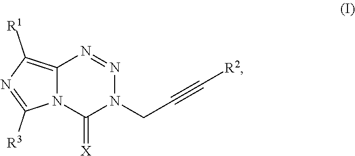
or a salt thereof;
wherein
X is O or S;
R¹ is —C(═O)NR^{b}R^{c}, F, Br, or I; wherein
R^{b} is —(C₁-C₆)alkyl, —(C₃-C₆)cycloalkyl, or H;
R^{c} is —(C₁-C₆)alkyl, —(C₃-C₆)cycloalkyl, or H;
R² is —(C₁-C₆)alkyl, —(C₃-C₆)cycloalkyl, or H; and
R³ is H, halo, —(C₁-C₆)alkyl, or —(C₃-C₆)cycloalkyl;
wherein each —(C₁-C₆)alkyl or —(C₃-C₆)cycloalkyl are optionally substituted with one or more substituents; and
each —(C₁-C₆)alkyl is unbranched or branched.

2. The compound of claim 1 wherein X is O.

3. The compound of claim 1 wherein R¹ is —C(═O)—N[(C₁-C₆)alkyl]₂ or —C(═O)—NH(C₁-C₆)alkyl.

4. The compound of claim 1 wherein R² is CH₃ or H.

5. The compound of claim 1 wherein R³ is H.

6. The compound of claim 1 wherein the compound of Formula I is a compound of Formula II:

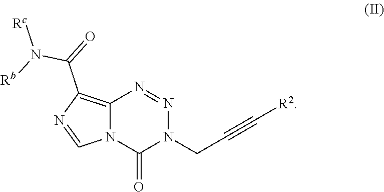

7. The compound of claim 6 wherein R^{b} is —(C₁-C₆)alkyl, R^{c} is —(C₁-C₆)alkyl, and R² is —(C₁-C₆)alkyl; or wherein R^{b}, R^{c} and R² are CH₃.

8. The compound of claim 1 wherein the compound is:

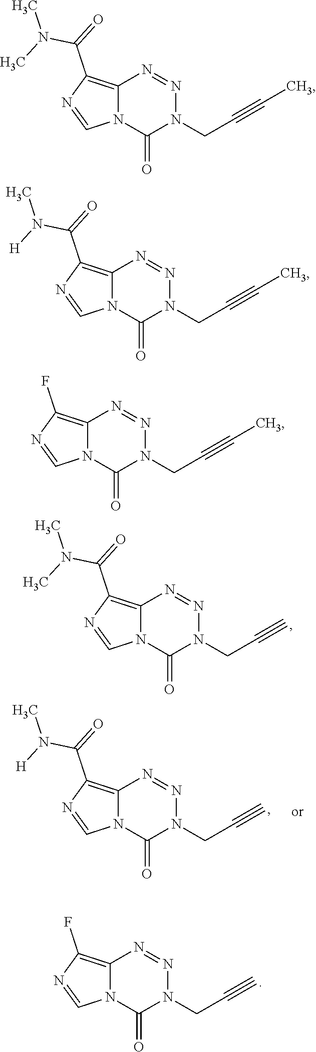

9. A composition comprising a compound of claim 1 and a pharmaceutically acceptable excipient.

10. A method of treating a cancer comprising administering to a cancer subject a therapeutically effective amount of a compound of claim 1 wherein the cancer is thereby treated.

11. The method of claim 10 wherein the cancer is melanoma, leukemia, breast cancer, lung cancer, pancreatic cancer, prostate cancer, colon cancer or brain cancer; or
wherein the cancer is glioblastoma, gliosarcoma, or meningioma.

12. The method of claim 10 wherein a dose of the compound administered is about 0.5 mg/kg to about 100 mg/kg of body weight per day; or
wherein a dose of the compound administered is 5 mg/m² to 1000 mg/m² of body surface area; or
wherein a unit dose of the compound administered is 5 mg to 1000 mg; or
wherein a therapeutically effective concentration of the compound contacting the cancer is about 1 nM to about 10 μM.

13. The method of claim 10 wherein a dose of the compound is administered once per day, twice per day, or thrice per day.

14. The method of claim 10 wherein the compound is administered orally, intravenously, or intracranially.

15. The method of claim 10 wherein the compound is 3-(but-2-yn-1-yl)-N,N-dimethyl-4-oxo-3,4-dihydroimidazo[5,1-d][1,2,3,5]tetrazine-8-carboxamide (TABZ).
